(19) United States
(12) Patent Application Publication
(10) Pub. No.: US 2013/0115628 A1
(43) Pub. Date: May 9, 2013

(54) METHOD FOR SELECTION OF CHEMOTHERAPEUTIC AGENTS FOR ADENOCARCINOMA CANCER

(71) Applicants: Jeffrey Dean Isaacson, Superior, CO (US); Mical Raponi, San Francisco, CA (US); James Ranger-Moore, Tucson, AZ (US); Eric L. Powell, Oceanside, CA (US)

(72) Inventors: Jeffrey Dean Isaacson, Superior, CO (US); Mical Raponi, San Francisco, CA (US); James Ranger-Moore, Tucson, AZ (US); Eric L. Powell, Oceanside, CA (US)

(73) Assignees: VENTANA MEDICAL SYSTEMS, INC., Tucson, AZ (US); CLOVIS ONCOLOGY, INC., Boulder, CO (US)

(21) Appl. No.: 13/565,283

(22) Filed: Aug. 2, 2012

Related U.S. Application Data
(60) Provisional application No. 61/514,160, filed on Aug. 2, 2011.
(60) Provisional application No. 61/514,168, filed on Aug. 2, 2011.
(60) Provisional application No. 61/514,173, filed on Aug. 2, 2011.
(60) Provisional application No. 61/514,182, filed on Aug. 2, 2011.
(60) Provisional application No. 61/514,937, filed on Aug. 4, 2011.
(60) Provisional application No. 61/525,322, filed on Aug. 19, 2011.
(60) Provisional application No. 61/525,327, filed on Aug. 19, 2011.
(60) Provisional application No. 61/525,329, filed on Aug. 19, 2011.
(60) Provisional application No. 61/525,343, filed on Aug. 19, 2011.
(60) Provisional application No. 61/525,352, filed on Aug. 19, 2011.
(60) Provisional application No. 61/525,360, filed on Aug. 19, 2011.
(60) Provisional application No. 61/547,856, filed on Oct. 17, 2011.
(60) Provisional application No. 61/651,766, filed on May 25, 2012.

(51) Int. Cl. G01N 33/68

(52) U.S. Cl. CPC G01N 33/68
USPC 435/7.23

(57) ABSTRACT

The subject invention relates to determining the presence and level of hENT1 expression in tumor tissue that is appropriate for gemcitabine therapy, and more importantly, the level of hENT1 expression that signifies that treatment with a gemcitabine derivative is a more appropriate strategy.

### CROSS-REFERENCE TO RELATED APPLICATIONS

This application claims priority under 35 U.S.C. §119(e) to U.S. Provisional Patent Application Ser. No. 61/514,160, which was filed on Aug. 2, 2011; to U.S. Provisional Patent Application Ser. No. 61/514,168, which was filed Aug. 2, 2011; to U.S. Provisional Patent Application Ser. No. 61/514,173, which was filed on Aug. 2, 2011; to U.S. Provisional Patent Application Ser. No. 61/514,182, which was filed Aug. 2, 2011; to U.S. Provisional Patent Application Ser. No. 61/514,937, which was filed Aug. 4, 2011; to U.S. Provisional Patent Application Ser. No. 61/525,322, which was filed Aug. 19, 2011; to U.S. Provisional Patent Application Ser. No. 61/525,327, which was filed Aug. 19, 2011; to U.S. Provisional Patent Application Ser. No. 61/525,329, which was filed Aug. 19, 2011; to U.S. Provisional Patent Application Ser. No. 61/525,343, which was filed Aug. 19, 2011; to U.S. Provisional Patent Application Ser. No. 61/525,352, which was filed Aug. 19, 2011; to U.S. Provisional Patent Application Ser. No. 61/525,360, which was filed Aug. 19, 2011; to U.S. Provisional Patent Application Ser. No. 61/547,856, which was filed Oct. 17, 2011; and to U.S. Provisional Patent Application Ser. No. 61/651,766, which was filed May 25, 2012, the disclosure of each of which is incorporated herein in its entirety.

In compliance with 37 C.F.R. §1.71(g)(1), disclosure is herein made that the invention(s) described and claimed were made pursuant to a Joint Research Agreement as that term is defined in 35 U.S.C. §103 (c)(3), that was in effect on or before the date the invention(s) were made, and as a result of activities undertaken within the scope of the Joint Research Agreement, by or on the behalf of Ventana Medical Systems, Inc. and Clovis Oncology, Inc.

### BACKGROUND OF THE INVENTION

Over 42,000 new cases of pancreatic cancer occurred in the U.S. in 2009 (American Cancer Society; Cancer Facts and Figures; 2009). The American Cancer Society estimates that 1 and 5 year overall survival (OS) rates are 24 and 5%, respectively. The majority of these patients either present with unresectable Stage III/IV disease, or tumor recurs after surgical resection and adjuvant chemoradiotherapy for Stage II or III disease. The standard palliative first-line therapy for patients with unresectable disease is gemcitabine monotherapy (Burris, H. A. et al. (1997) J. Clin. Oncol. 15(6):2403-2413). Unfortunately, many of these patients fail to derive substantial benefit from treatment—median OS in Stage III/IV disease is approximately 5-7 months (Burris et al., supra; Herrmann, Y. et al. J. Clin. Oncol. 2007 ASCO Ann. Mtg. Proc. (Post-Mtg. Ed.); 25(18S (June 20 Suppl)):15187). No clinical or molecular marker is established to predict benefit from gemcitabine therapy; therefore, patients are treated empirically until evidence of disease progression or worsening performance status.

Recently, attention has turned to candidate molecular markers in the tumor tissue that may predict outcome. Gemcitabine is a highly hydrophilic nucleoside analogue that enters cells only through specific membrane transporters. Expression of several transporters on pancreatic tumor cells has been examined in relationship to clinical outcome. The human equilibrative nucleoside transporter-1 (hENT1), in particular, has been shown by multiple groups to be a predictive marker of survival after gemcitabine chemotherapy. hENT1 is a high-flux transporter, expressed variably on pancreatic tumor cells, and the hypothesis is that low expression of hENT1 impairs drug entry into tumor cells and is thus associated causally with poor gemcitabine outcome.

The subject invention addresses the issue of the presence and level of hENT1 expression in tumor tissue that is appropriate for gemcitabine therapy, and more importantly, the level of hENT1 expression that signifies that treatment with a gemcitabine derivative is a more appropriate strategy.

### SUMMARY OF THE INVENTION

In one embodiment, the subject invention relates to a method for treatment of cancer in an individual that includes receiving assay results that the level of hENT1 protein of said cancer is below a predetermined level, and administering a therapeutic agent comprising a gemcitabine derivative.

In one embodiment, the subject invention relates to a therapeutic agent comprising a gemcitabine derivative for use in the treatment of cancer in an individual having tumor tissue with a level of hENT1 protein classified as Low, wherein said gemcitabine derivative is capable of being transported into the tumor tissue by a mechanism that is independent of the hENT1 protein, wherein said level of hENT1 protein is determined in an immunohistochemistry assay in said tumor tissue, and wherein said Low classification meets the criterion of having less than 50% of the tumor tissue display hENT1 membrane staining with a hENT1 antibody upon examination with a 10× ocular of a light microscope.

In one embodiment, the subject invention relates to the use of a therapeutic agent comprising a gemcitabine derivative for the preparation of a medicament for the treatment of cancer in an individual having tumor tissue with a level of hENT1 protein classified as Low, wherein said gemcitabine derivative is capable of being transported into the tumor tissue by a mechanism that is independent of the hENT1 protein, wherein said level of hENT1 protein is determined in an immunohistochemistry assay in said tumor tissue, and wherein said Low classification meets the criterion of having less than 50% of the tumor tissue display hENT1 membrane staining with a hENT1 antibody upon examination with a 10× ocular of a light microscope.

In one embodiment, the cancer is pancreatic adenocarcinoma.

The gemcitabine derivative can comprise the compound of formula I:

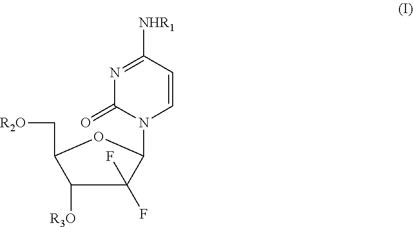

wherein R₁ and R₃ are hydrogen and R₂ is a C₁₈- or C₂₀-saturated and monounsaturated acyl group, or a pharmaceutically acceptable salt thereof. In one embodiment, the gemcitabine derivative is Gemcitabine-5′-Elaidate.

In one embodiment, the Gemcitabine-5′-Elaidate can be transported through the plasma membrane by a mechanism that does not utilize the hENT1 transporter. The mechanism of transport can be by passive diffusion across the membrane.

The assay method for the determination of the hENT1 transporter can be an immunoassay. In a preferred embodiment, the immunoassay can be an immunohistochemistry assay.

In one embodiment, the assay result that the level of hENT1 protein is below a predetermined level is specified as Low by Method Six. A sample is defined as Low when the sample has the criterion of less than 50% of the tumor tissue displays hENT1 membrane staining with use of a 10× ocular using a light microscope (100× total magnification). A sample is defined as High when the sample has the criterion of at least 50% of the tumor displays hENT1 membrane staining with use of a 10× ocular using a light microscope (100× total magnification).

In one embodiment, the assay result that the level of hENT1 protein is below a predetermined level is specified as Low by Method Six. A Low classification is specified if either:
a) the percent of membrane intensity classified as 0 is greater than 50%, or
b) there is no definitely positive (PP) tumor tissue observed at 10× magnifications.

A High classification is specified if both:
a) the percent of membrane intensity classified as 0 is less than or equal to 50%, and
b) a definitely positive (PP) tumor tissue is observed at magnifications of 2×, 4×, and/or 10×.

If multiple samples from the same patient are obtained, if any of the samples are classified as High, then that patient is classified as High.

In one embodiment, where it is found that the patient has hENT1 protein levels that are Low as defined above, he can be administered the gemcitabine derivative.

In one embodiment, the subject invention relates to a method for treatment of cancer in an individual that includes administering a therapeutic agent comprising a gemcitabine derivative to said individual, wherein said cancer has previously been identified as a cancer which has hENT1 protein below a predetermined level.

In one embodiment, the subject invention relates to a method for treatment of cancer in an individual comprising: a) causing an assay to be conducted for the level of hENT1 transporter in cancer cells from said individual to determine whether the hENT1 protein level is below a predetermined level; and b) if said assay indicates that hENT1 protein is below a predetermined level, administering a therapeutic agent comprising a gemcitabine derivative.

In one embodiment, the subject invention relates to a method for determining whether a gemcitabine derivative is suitable for administration to a patient with cancer, comprising the steps of: a) causing an assay to be conducted for the level of hENT1 protein in cancer cells from said patient to determine whether the hENT1 protein level is below a predetermined level; b) selecting for a patient having hENT1 protein below the predetermined level, and c) administering a therapeutic agent comprising the gemcitabine derivative.

In one embodiment, the subject invention relates to a method comprising administering a pharmaceutically effective amount of a gemcitabine derivative to a subject in need of a cancer treatment, wherein the subject has been identified as being susceptible to treatment with gemcitabine derivative by: a) obtaining a sample derived from the patient, and b) causing the level of hENT1 protein in said sample to be determined, wherein the subject is susceptible when the hENT1 protein is below a predetermined level.

In one embodiment, the subject invention relates to the use of a gemcitabine derivative for the preparation of a medicament for treating cancer in an individual wherein said cancer has previously been identified as a cancer which has hENT1 protein below a predetermined level.

In one embodiment, the subject invention relates to the preparation of a gemcitabine derivative for the treatment of cancer, comprising formulating said gemcitabine derivative for the treatment of cancer in an individual, wherein the cancer has previously been identified as a cancer which has hENT-1 protein below a predetermined level.

In one embodiment, the subject invention relates to a gemcitabine derivative for use in the treatment of cancer, wherein said gemcitabine derivative is capable of being transported into the tumor tissue by a mechanism that is independent of the hENT1 protein, and further wherein the cancer has previously been identified as a cancer which has hENT-1 protein classified as Low, and wherein said Low classification meets the criterion of having less than 50% of the cancer cells display hENT1 membrane staining with a hENT1 antibody upon examination with a 10× ocular of a light microscope.

In one embodiment, the subject invention relates to the use of a gemcitabine derivative for the preparation of a medicament for the treatment of cancer, wherein said gemcitabine derivative is capable of being transported into the tumor tissue by a mechanism that is independent of the hENT1 protein, and further wherein the cancer has previously been identified as a cancer which has hENT-1 protein classified as Low, and wherein said Low classification meets the criterion of having less than 50% of the cancer cells display hENT1 membrane staining with a hENT1 antibody upon examination with a 10× ocular of a light microscope.

In one embodiment, the subject invention relates to a therapeutic agent comprising a gemcitabine derivative for use in the treatment of cancer in an individual, wherein said gemcitabine derivative is capable of being transported into the tumor tissue by a mechanism that is independent of the hENT1 protein, wherein said cancer has previously been identified as a cancer having a level of hENT1 protein classified as Low, and wherein said Low classification meets the criterion of having less than 50% of the tumor tissue display hENT1 membrane staining with a hENT1 antibody upon examination with a 10× ocular of a light microscope.

In one embodiment, the subject invention relates to the use of a therapeutic agent comprising a gemcitabine derivative for the preparation of a medicament for the treatment of cancer in an individual, wherein said gemcitabine derivative is capable of being transported into the tumor tissue by a mechanism that is independent of the hENT1 protein, wherein said cancer has previously been identified as a cancer having a level of hENT1 protein classified as Low, and wherein said Low classification meets the criterion of having less than 50% of the tumor tissue display hENT1 membrane staining with a hENT1 antibody upon examination with a 10× ocular of a light microscope.

In one embodiment, the subject invention relates to an effective amount of a gemcitabine derivative for use in the treatment of cancer in a subject in need of a cancer treatment, wherein the gemcitabine derivative is capable of being transported into the tumor tissue by a mechanism that is independent of the hENT1 protein, wherein the subject has been identified as being susceptible to treatment with gemcitabine derivative by determining the level of hENT1 protein in a biological sample from the patient, wherein the subject is susceptible when the hENT1 protein level is classified as Low, and wherein said Low classification meets the criterion of having less than 50% of the cancer cells display hENT1 membrane staining with a hENT1 antibody upon examination with a 10× ocular of a light microscope.

In one embodiment, the subject invention relates to the use of an effective amount of a gemcitabine derivative for the preparation of a medicament for the treatment of cancer in a subject in need of a cancer treatment, wherein the gemcitabine derivative is capable of being transported into the tumor tissue by a mechanism that is independent of the hENT1 protein, wherein the subject has been identified as being susceptible to treatment with gemcitabine derivative by determining the level of hENT1 protein in a biological sample from the patient, and wherein the subject is susceptible when the hENT1 protein level is classified as Low, and wherein said Low classification meets the criterion of having less than 50% of the cancer cells display hENT1 membrane staining with a hENT1 antibody upon examination with a 10× ocular of a light microscope.

In one embodiment, the subject invention relates to a method for determining whether a gemcitabine derivative is suitable for administration to a patient with cancer, comprising the steps of: a) obtaining a biological sample comprising cancer cells or cancer cell proteins derived from said patient; b) conducting an assay for hENT1 protein on said biological sample to determine whether the hENT1 protein level is below a predetermined level; and c) providing results of said assay to a healthcare professional wherein said healthcare professional administers a therapeutic agent comprising said gemcitabine derivative if said assay indicates that the level of hENT1 protein of said cancer is below a predetermined level.

In one embodiment, the subject invention relates to a method for determining whether a gemcitabine derivative that is capable of being transported into the tumor tissue by a mechanism that is independent of the hENT1 protein is suitable for administration to a patient with cancer, comprising conducting an immunohistochemistry assay on a biological sample comprising cancer cells or cancer cell proteins from said patient to determine whether the hENT1 protein level is classified as Low, wherein said Low classification meets the criterion of having less than 50% of the cancer cells display hENT1 membrane staining with a hENT1 antibody upon examination with a 10× ocular of a light microscope, wherein if said assay indicates that the level of hENT1 protein is Low, the gemcitabine derivative that is capable of being transported into the tumor tissue by a mechanism that is independent of the hENT1 protein is suitable for administration to a patient with cancer.

In one embodiment, the subject invention relates to a method for determining whether a gemcitabine derivative is suitable for administration to a patient with cancer. This determination includes the steps of: (a) conducting an assay for hENT1 protein in cancer cells to determine whether the hENT1 protein level is below a predetermined level; (b) selecting for the patient having hENT1 protein below the predetermined level; and (c) administering a therapeutic agent comprising the gemcitabine derivative. Following this method, it has been found that the administration of the gemcitabine derivative can be effective in the treatment of the patient's cancer.

In one embodiment, the subject invention relates to a method for determining whether a gemcitabine derivative is suitable for administration to a patient with pancreatic adenocarcinoma. This determination includes the steps of: (a) conducting an assay for hENT1 protein in adenocarcinoma cells to determine whether the hENT1 protein level is below a predetermined level; (b) selecting for the patient having hENT1 protein below the predetermined level; and (c) administering a therapeutic agent comprising the gemcitabine derivative. Following this method, it has been found that the administration of the gemcitabine derivative can be effective in the treatment of the patient's pancreatic adenocarcinoma.

In a further embodiment, the invention is directed to a method for the treatment of cancer in a patient with a gemcitabine derivative. This method comprises the steps of: (a) determining whether hENT1 is below a predetermined level in cancer cells from a patient; and (b) administering to a patient having hENT1 below the predetermined level, a therapeutically effective amount of the gemcitabine derivative.

In a further embodiment, the invention is directed to a method for the treatment of pancreatic adenocarcinoma in a patient with a gemcitabine derivative. This method comprises the steps of: (a) determining whether hENT1 is below a predetermined level in adenocarcinoma cells from a patient; and (b) administering to a patient having hENT1 below the predetermined level, a therapeutically effective amount of the gemcitabine derivative.

In one embodiment, the subject invention relates to a system for treatment of cancer in an individual, said system comprising a facility for receiving assay results indicating that the level of hENT1 protein of said cancer is below a predetermined level, and a facility for administering a therapeutic agent comprising a gemcitabine derivative in response to said assay results.

In one embodiment, the subject invention relates to a system for the formulation and distribution of a gemcitabine derivative for the treatment of cancer, said system comprising a facility for formulating said gemcitabine derivative, and a facility for distributing said gemcitabine derivative to healthcare providers for administration of the gemcitabine derivative to a cancer patient, wherein the cancer has previously been identified as a cancer which has hENT-1 protein below a predetermined level.

In one embodiment, the predetermined level of hENT1 is an assay result having a hENT1 classification by Method Two that is Negative (N), or a classification that is specified by the magnification at which the hENT1 staining is definitely positive (PP) at 20×, or 40×, but not at 2×, 4× or 10×.

In one embodiment, where it is found that the patient has hENT1 expression that is N or is only definitely positive (PP) at magnifications for 20× or 40×, he can be administered the gemcitabine derivative.

If multiple samples from the same patient are obtained, the lowest magnifiation (i.e. 2×<4×<10×<20×<40×) when definitely positive (PP) is obtained from the evaluations is used.

In one embodiment, the predetermined level of hENT1 is an assay result having a hENT1 classification by Method Five that provides a percent of membrane intensity classified as 0 that is greater than 50% (No hENT1).

In one embodiment, where it is found that the patient has a percent of membrane intensity classified as 0 that is greater than 50%, he can be administered the gemcitabine derivative.

If multiple samples from the same patient are obtained, if any of the samples are classified as Low or High hENT1 (as defined by Method Five), then the patient cannot be classified as No hENT1.

In one embodiment, the predetermined level of hENT1 is an assay result having a hENT1 classification by Method One that provides an H-score that is less than 20, or an H-score that is less than 50, or an H-score that is less than 80. An H-score of less than 50 is preferred.

In one embodiment, where it is found that the patient has an H-Score of 20, he can be administered the gemcitabine derivative. In an alternative, a patient having an H-Score of less than 50 can also be administered the gemcitabine derivative. In an alternative, a patient having an H-score less than 80 can also be administered the gemcitabine derivative. Administering a gemcitabine derivative to a patient having an H-Score of less than 50 is preferred.

If multiple samples from the same patient are obtained, the highest H-score from all samples is the H-score for that patient.

In one embodiment, the predetermined level of hENT1 is an assay result having a hENT1 classification by Method Three that provides no membrane staining and is referred to as Negative (N)'. In one embodiment, the predetermined level of hENT1 is an assay result having a hENT1 classification by Method Three that provides no membrane staining or a few areas of positive staining and is referred to as Negative (N) or Segmental (S), respectively.

In one embodiment, where it is found that the patient has no membrane staining, he can be administered the gemcitabine derivative. In an alternative, a patient having no membrane staining or a few areas of positive staining can also be administered the gemcitabine derivative.

If multiple samples from a patient are obtained, the maximum result (i.e., D>S>N) from the evaluations is used.

In one embodiment, the predetermined level of hENT1 is an assay result having a Negative (N) hENT1 staining intensity.

In one embodiment, a patient having a Negative (N) hENT1 staining intensity can be administered the gemcitabine derivative. In the alternative, a patient having a Negative (N) or Weak (W) hENT1 staining intensity can be administered the gemcitabine derivative.

In one embodiment, a patient having No hENT1 as defined by Method Six can be administered the gemcitabine derivative.

In one embodiment, a method of classifying hENT1 biomarker expression in a tissue sample comprises: a. obtaining a tissue sample from a cancer patient; b. visualizing hENT1 biomarker protein expression in the tissue sample using immunohistochemical staining with an anti-hENT1 antibody; c. determining hENT1 protein staining intensity in the tissue sample; and e. classifying hENT1 biomarker expression as LOW when less than 50% of the tissue sample displays hENT1 membrane staining with an anti-hENT1 antibody upon examination with a 10× ocular of a light microscope.

In one embodiment, a method of identifying a pancreatic cancer patient suitable for treatment with a gemcitabine derivative, comprises: a. visualizing hENT1 protein expression in cancer cells from the patient by immunohistochemical staining using an anti-hENT1 antibody; b. assigning a protein expression classification of LOW when less than 50% of the cancer cells display hENT1 membrane staining upon examination with a 10× ocular of a light microscope; and c. identifying patients exhibiting LOW hENT1 protein expression as being suitable for treatment with a gemcitabine derivative.

In one embodiment, the subject invention relates to a method of identifying a pancreatic cancer patient suitable for treatment with a gemcitabine derivative, comprising: visualizing hENT1 protein expression in cancer cells in a biological sample from the patient by immunohistochemical staining using an anti-hENT1 antibody, and assigning a protein expression classification of LOW when less than 50% of the cancer cells display hENT1 membrane staining upon examination with a 10× ocular of a light microscope, wherein if a protein expression classification is assigned LOW, the pancreatic cancer patient is suitable for treatment with a gemcitabine derivative.

In one embodiment, a method of predicting a pancreatic cancer patient's responsiveness to gemcitabine therapy comprises: a. obtaining a tissue sample from the cancer patient; b. visualizing hENT1 biomarker protein expression in the tissue sample using immunohistochemical staining with an anti-hENT1 antibody; c. determining hENT1 protein staining intensity in the tissue sample; d. classifying hENT1 biomarker expression as LOW when less than 50% of the tissue sample displays hENT1 membrane staining with an anti-hENT1 antibody upon examination with a 10× ocular of a light microscope; and e. predicting a poor response to gemcitabine therapy when the patient's tissue sample is classified as having LOW hENT1 biomarker expression.

In one embodiment, the subject invention relates to a method of predicting a pancreatic cancer patient's responsiveness to gemcitabine therapy comprising visualizing hENT1 biomarker protein expression in a tissue sample from the pancreatic cancer patient using immunohistochemical staining with an anti-hENT1 antibody, determining hENT1 protein staining intensity in the tissue sample, classifying hENT1 biomarker expression as LOW when less than 50% of the tissue sample displays hENT1 membrane staining with an anti-hENT1 antibody upon examination with a 10× ocular of a light microscope, and predicting a poor response to gemcitabine therapy when the patient's tissue sample is classified as having LOW hENT1 biomarker expression.

In one embodiment, a method of stratifying cancer patients for overall survival comprises: a. staining tumor tissue with an anti-hENT1 antibody; b. visually detecting antibody bound to the tumor tissue; c. scoring antibody staining intensity; and d. classifying the cancer patients into a LOW hENT1 expression subgroup when greater than 50% of the tumor tissue is scored as negative for hENT1 membrane staining with a membrane intensity of 0; and e. stratifying the LOW hENT1 expression subgroup as having shorter overall survival as compared with subgroups that are not classified as exhibiting LOW hENT1 expression.

In one embodiment, the subject invention relates to a method of stratifying cancer patients for overall survival comprising: staining a biological sample comprising tumor tissue of each cancer patient with an anti-hENT1 antibody, visually detecting antibody bound to the tumor tissue, scoring antibody staining intensity, and classifying the cancer patient into a LOW hENT1 expression subgroup when greater than 50% of the tumor tissue is scored as negative for hENT1 membrane staining with a membrane intensity of 0, and stratifying the LOW hENT1 expression subgroup as having shorter overall survival as compared with subgroups that are not classified as exhibiting LOW hENT1 expression.

### BRIEF DESCRIPTION OF THE FIGURES

FIG. 1 depicts an example of a score sheet. Such a score sheet is particularly useful for the scoring algorithms used in methods one through six. N means negative. P means positive, PP means definitely positive, NA means not applicable, D means diffuse, and S means segmented.

FIG. 2 depicts an example of a score sheet. Such a score sheet is particularly useful for the scoring algorithm used in method six.

FIG. 3 depicts the frequency of patients with hENT1 classification in subgroups of High and Low hENT1 protein levels as described in method six where a sample is defined as High when the sample has the criterion of at least 50% of the tumor displays hENT1 membrane staining with use of a 10× ocular using a light microscope (100× total magnification) and a sample is defined as Low when the sample has the criterion of less than 50% of the tumor tissue displays hENT1 membrane staining with use of a 10× ocular using a light microscope (100× total magnification).

FIG. 4 is a Kaplan-Meier plot of overall survival (OS) for gemcitabine patients in subgroups of High (1) and Low (2) hENT1 expression as determined using the method six algorithm described in FIG. 3.

FIG. 5 is a Kaplan-Meier plot of overall survival for 5-FU Patients in subgroups of High (1) and Low (2) hENT1 expression as determined using the method six algorithm described in FIG. 3.

FIG. 6 depicts the frequency of patients with hENT1 classification in subgroups of High and Low hENT1 expression as described in method six where a sample is defined as High when the sample is determined to have: a) definitely positive (PP) tumor tissue observed at a magnification of 2×, 4×, and/or 10×, AND b) the percent of negative tumor tissue (Membrane intensity classified as 0) is <=50%. A sample is defined as Low when the sample is determined to have either: a) >50% of negative tumor tissue (Membrane intensity is 0) OR b) definitely positive (PP) tumor tissue observable only at a magnification level of 20× or 40× or not at any of the tested magnifications. If multiple samples were obtained from a patient, if any of the samples were classified as High, then that patient is classified as High.

FIG. 7 is a Kaplan-Meier plot of overall survival (OS) for gemcitabine patients in subgroups of High (1) and Low (2) hENT1 expression as determined using the method six algorithm described in FIG. 6.

FIG. 8 is a Kaplan-Meier plot of overall survival for 5-FU Patients in subgroups of High (1) and Low (2) hENT1 expression as determined using the method six algorithm described in FIG. 6.

FIG. 9 depicts that the concordance of hENT1 expression between matched primary and metastatic tissue samples taken from the same patient as determined by method six.

FIG. 10 is a Kaplan-Meier plot of OS for patients treated with gemcitabine and hENT1 status (High [1] and Low [2]) based on primary tumor tissue. The hENT1 expression was determined using the method six algorithm described in FIG. 3.

FIG. 11 is a Kaplan-Meier plot of OS for patients never treated with gemcitabine and hENT1 status (High [1] and Low [2]) based on primary tumor tissue. The hENT1 expression was determined using the method six algorithm described in FIG. 3.

FIG. 12 is a Kaplan-Meier plot of OS for patients treated with gemcitabine and hENT1 status (High [1] and Low [2]) based on metastatic tumor tissue. The hENT1 expression was determined using the method six algorithm described in FIG. 3.

FIG. 13 is a Kaplan-Meier plot of OS for patients never treated with gemcitabine and hENT1 status (High [1] and Low [2]) based on metastatic tumor tissue. The hENT1 expression was determined using the method six algorithm described in FIG. 3.

FIG. 14 depicts the frequency of patients with hENT1 classification in subgroups of 2×, 4×, 10×, 20×, 40×, N as described in method two.

FIG. 15 is a Kaplan-Meier plot of overall survival (OS) for gemcitabine patients in subgroups of N, 20× or 40× vs 2×, 4×, or 10× as described in method two.

FIG. 16 is a Kaplan-Meier plot of overall survival for 5-FU Patients in subgroups of N, 20× or 40× vs 2×, 4×, or 10× as described in method two.

FIG. 17 depicts the frequency of patients with hENT1 classification in the No, Low and High hENT1 expression subgroups as described in method five.

FIG. 18 depicts the median overall survival for gemcitabine and 5FU across subgroups of hENT1 expression defined by No hENT1, Low hENT1 and High hENT1.

FIG. 19 is a Kaplan-Meier plot of overall survival (OS) for gemcitabine patients in increasing subgroups of hENT1 expression as described in method five.

FIG. 20 is a Kaplan-Meier plot of overall survival (OS) for gemcitabine patients with No hENT1 expression compared to the combined subgroups of Low and High hENT1 expression as described in method five.

FIG. 21 depicts the frequency of patients with maximum H-score in increasing subgroups of H-score as described in method one.

FIG. 22 depicts the median overall survival for gemcitabine and 5FU across increasing subgroups of H-score as described in method one.

FIG. 23 is a Kaplan-Meier plot of overall survival (OS) for gemcitabine patients in increasing subgroups of H-score as described in method one.

FIG. 24 is a Kaplan-Meier plot of overall survival (OS) for gemcitabine patients with H-score<50 vs H-score>=50 as described in method one.

FIG. 25 depicts the frequency of patients across the Subgroups of hENT1 Expression Defined by Negative (N), Segmental (S), and Diffuse (D) as described in method three.

FIG. 26 depicts the median overall survival for gemcitabine and 5FU based on hENT1 staining pattern as described in method three.

FIG. 27 is a Kaplan-Meier plot of overall survival (OS) for gemcitabine patients across subgroups defined by staining pattern as described in method three.

FIG. 28 is a Kaplan-Meier plot of overall survival (OS) for gemcitabine patients with a negative staining pattern compared to the combined subgroups of Segmental and Diffuse staining pattern as described in method three.

FIG. 29 depicts the frequency of patients with hENT1 classification in subgroups of Negative, Weak, Moderate, and Strong as described in method four.

FIG. 30 depicts the median overall survival for gemcitabine and 5FU based on hENT1 staining pattern as described in method four.

FIG. 31 is a Kaplan-Meier plot of overall survival (OS) for gemcitabine patients in increasing subgroups of N, W, or M as described in method four.

FIG. 32 depicts the frequency of patients across the 3 subgroups of hENT1 expression defined categorically as High, Mid, and No, where a sample is defined as High when the sample has the criterion based on the exemplary scoring sheet of FIG. 2 of Yes for ≧50% tumor cells membrane resolvable at 10×, a sample is defined as Mid when the sample has the criterion of Yes for membranes resolvable at 10× and No for ≧50% tumor cells membrane resolvable at 10×, and a sample is defined as No when the sample has the criterion of No for membranes resolvable at 10× and No for ≧50% tumor cells membrane resolvable at 10×. If multiple samples were obtained from a patient, if any of the samples were classified as High, then that patient is classified as High.

FIG. 33 illustrates that in gemcitabine-treated patients, the subgroup with No hENT1 expression exhibited the shortest OS followed by the Mid hENT1 subgroup and the High hENT1 subgroup had the longest OS.

FIG. 34 shows, in contrast to FIG. 33, that 5FU treated patients do not demonstrate improvement in OS between the High, Mid and No hENT1 subgroups of patients.

### DETAILED DESCRIPTION OF THE INVENTION

Pancreatic cancer is a very serious form of cancer. The majority of patients present with unresectable disease, and the condition is often not diagnosed until the cancer is relatively advanced. The standard first-line treatment for patients with unresectable pancreatic cancer is gemcitabine monotherapy. Unfortunately many of these patients fail to derive benefit from this treatment. No clinical or molecular marker has been established to predict benefit from gemcitabine therapy, so patients are treated empirically until evidence of disease progression or worsening performance status.

It is a main objective of the present invention to provide a cut-off point or range of cut-off points for hENT1 protein level in the patient's pancreatic adenocarcinoma cells at which it is clear that gemcitabine is unlikely to be effective as a first line treatment. This cut-off point informs the health practitioner that alternative therapy is appropriate. In particular, the cut-off point indicates when gemcitabine derivative therapy can be appropriate and effective. The cut-off also determines when gemcitabine therapy is appropriate, (i.e., at hENT1 expression levels above the hENT1 cut-off, gemcitabine is most likely to be effective).

It is also recognized by the inventors that even in circumstances where the hENT1 protein level is above the cut-off point and the patient is receiving gemcitabine therapy, it can be appropriate to utilize as an adjunctive or replacement therapy, the gemcitabine derivative in order to improve OS.

To assist in the understanding, explanation and practice of the subject invention, the definitions of terms are provided throughout the Detailed Description.

As used herein, “cut-off” or “cut-off value” refers to a single value or range of values for hENT1 transporter protein expression in adenocarcinoma cells below which gemcitabine is not or is not likely to be effective in improving the overall survival (OS) of the patient. Cut-off can be expressed either qualitatively, i.e., as in the presence or absence of hENT1 protein in the cells, or quantitatively, as an H-score. The term “predetermined level” is synonymous with the cut-off qualitative or quantitative value, and is the standard against which the hENT1 protein level in the patient's adenocarcinoma cells is compared.

An “H-score” is defined in detail below.

“hENT” is an acronym for human equilibrative nucleoside transporter. The equilibrative nucleoside transporter (ENT) family, also known as SLC29, is a group of plasmalemnal transport proteins which transport nucleoside substrates such as adenosine into cells. There are four known ENTs, designated ENT1, ENT2, ENT3, and ENT4. ENTs are blocked by adenosine reuptake inhibitors such as dipyridamole and dilazep. The concentrative nucleoside transporter (CENT) family, also known as SLC28, has three members: SLC28A1, SLC28A2 and SLC28A3, also designated as CNT1, CNT2 and CNT3.

“hENT1” is a protein that in humans is encoded by the SLC29A1 gene. This transmembrane glycoprotein localizes to at least the plasma and mitochondrial membranes and mediates the cellular uptake of nucleosides from the surrounding medium. Nucleoside transporters generally are required for nucleotide synthesis in cells that lack de novo nucleoside synthesis pathways, and are also necessary for the uptake of cytotoxic nucleosides used for cancer and viral chemotherapies.

The entry of gemcitabine into tumor cells is dependent upon the expression of specific membrane transporter proteins, particularly hENT1. The hENT1 protein level in various tissues, cells and cell components is determined by methods described herein. The hENT1 level varies across populations of pancreatic adenocarcinoma patients from overexpression of the protein to no or little hENT1 expression. As is discussed herein, the level of hENT1 can be determined by immunoassays, immunohistochemistry, and the like.

“hENT1 antibody” refers to any antibody that specifically binds to hENT1 protein. The term “antibody” as used herein includes all forms of antibodies, including but not limited to recombinant antibodies, chimeric antibodies, single chain antibodies, humanized antibodies, fusion proteins, monoclonal antibodies, polyclonal antibodies, non-human antibodies, fully human antibodies, and antibody fragments. The modifier “monoclonal” indicates the character of the antibody as being obtained from a substantially homogeneous population of antibodies, and is not to be construed as requiring production of the antibody by any particular method. For example, monoclonal antibodies useful for methods described herein can be made by the hybridoma method first described by Kohler et al., Nature, 256:495 (1975), or can be made by recombinant DNA methods (see, e.g., U.S. Pat. No. 4,816,567). The “monoclonal antibodies” can also be isolated from phage antibody libraries using the techniques described in Clackson et al., Nature, 352:624-628 (1991) and Marks et al., J. Mol. Biol., 222:581-597 (1991). hENT1 antibody clone SP120 was developed by Spring Bioscience, a subsidiary of Ventana Medical Systems.

Detection of the Ab:hENT1 complex can be accomplished directly or indirectly using methods known in the art. In direct detection methods, the antibody further comprises a detectable label and unreacted antibodies can be removed from the complex. The amount of remaining label thereby indicates the amount of complex formed. It is preferable to select labels that remain attached to the agents even during stringent washing conditions. It is also preferable that the label not interfere with the binding reaction. In an indirect detection procedure, the label is introduced either chemically or enzymatically. A desired label generally does not interfere with binding or the stability of the resulting label:protein complex. However, the label is typically designed to be accessible to antibody for an effective binding and hence generating a detectable signal.

A wide variety of labels suitable for detecting protein levels are known in the art. Non-limiting examples include radioisotopes, enzymes, colloidal metals, fluorescent compounds, bioluminescent compounds, and chemiluminescent compounds.

The amount of labeled antibody:protein complexes formed during the binding reaction can be quantified by standard quantitative procedures known in the art. Such techniques include but are not limited to immunohistochemistry assays, radioimmunoassay, ELISA (enzyme-linked immunosorbent assay), “sandwich” immunoassays, immunoradiometric assays, in situ immunoassays (using. e.g., colloidal gold, enzyme or radioisotope labels), western blot analysis, immunoprecipitation assays, immunofluorescent assays, and SDS-PAGE.

Intensity of staining with a hENT1 antibody can be determined by any method known in the art and includes, but is not limited to, subjective analysis by visual inspection, and automated systems coupled with algorithms.

Magnification is used in some embodiments of the invention. Standard microscope techniques are well known in the art.

“Immunohistochemistry (IHC)” refers to a method of determining the presence or distribution of an antigen (such as a protein) in a sample (such as a pancreatic cancer sample, for example, a portion or section of tissue) by detecting interaction of the antigen with a specific binding agent, such as an antibody. A sample including an antigen (such as a target antigen) is incubated with an antibody under conditions permitting antibody-antigen binding. Antibody-antigen binding can be detected by means of a detectable label conjugated to the antibody (direct detection) or by means of a detectable label conjugated to a secondary antibody, which is raised against the primary antibody (e.g., indirect detection). Exemplary detectable labels that can be used for IHC include, but are not limited to, radioactive isotopes, fluorochromes (such as fluorescein, fluorescein isothiocyanate, and rhodamine), haptens, enzymes (such as horseradish peroxidase or alkaline phosphatase), and chromogens (such as 3,3′-diaminobenzidine or Fast Red). In some examples, IHC is utilized to detect the presence of or determine the amount of one or more proteins in a sample, for example, a pancreatic cancer sample.

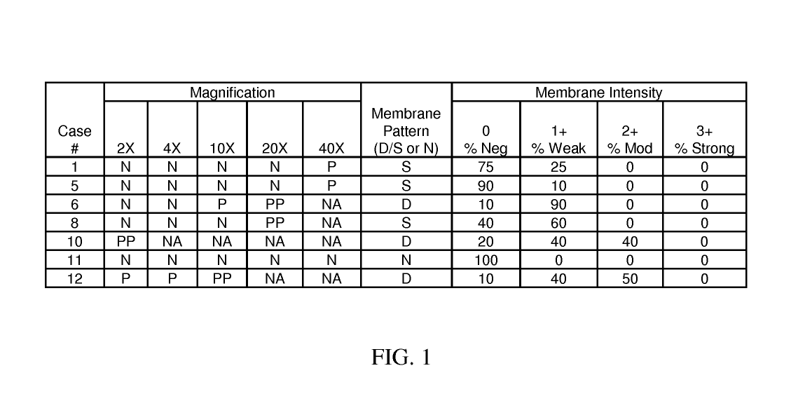

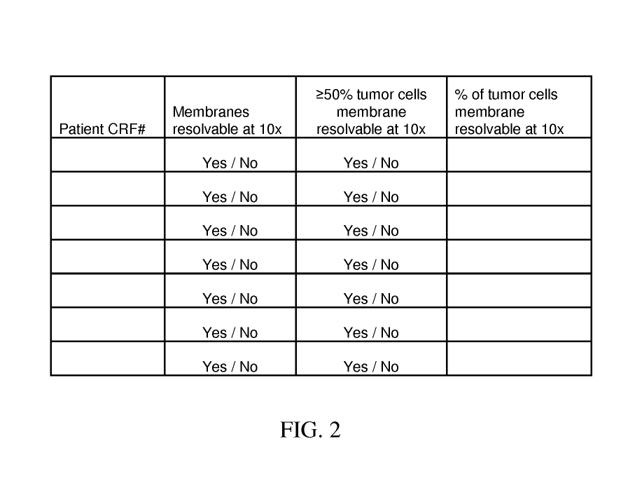

The term “hENT1 endpoint” refers to a classification of hENT1 expression for any sample of the invention. A hENT1 endpoint can be determined by multiple methods, but is generally a continuum that takes into account percentage of cells that are positive for the hENT1 protein and/or the intensity of staining with a hENT1 antibody. A scoring algorithm was implemented using a scoring sheet similar to the sheet included as FIGS. 1 and 2. Patients may have multiple hENT1 expression evaluations due to multiple cores of tumor tissue available per patient. For example, a patient may have 3 tissue cores that are each read by a pathologist. For each endpoint method provided below, the method for determining the result based on multiple evaluations is also provided.

Method One for determining a hENT1 endpoint is an H-score. The H-score is a continuous variable that takes into account both the percentage of cells that are positive for the antigen in question, as well as the intensity of staining with the relevant antibody (McCarty et al. (1986) Cancer Research (Supp) 46, 4244s-4248s). A scoring algorithm was used to capture the hENT1 expression results.

The immunostaining intensity of the tumor tissue has been scored on a 0, 1+, 2+, 3+ scale with the most intense staining given a score of 3+ and the absence of staining is scored a 0. The percentage of the tumor tissue displaying each of the 4 levels of immunostaining intensity is combined with the immunostaining intensity as follows:

H score=0* % of staining scored as a 0)+

1*(% of staining scored as a 1+)+

2*(% of staining scored as a 2+)+

3*(% of staining scored as a 3+)

The H-score ranges from 0 to 300 with 0 representing no staining and 300 representing the maximum intensity throughout all of the tumor tissue. if there are multiple evaluations made on a patient, the maximum result (i.e., highest H-score) from the evaluations is used. There may be circumstances where if multiple evaluations are made on a patient, the minimum result from the evaluations is used.

Method Two for determining a hENT1 endpoint is based on magnification. The hENT1 staining intensity is assessed at the following microscope magnifications: 2×, 4×, 10×, 20×, and 40×. At each level of magnification the presence of hENT1 staining in a tissue core is rated as negative (N), possibly positive (P), and definitively positive (PP). Once a magnification level is rated as PP, no higher levels of magnifications are monitored and the score is given as NA (see case #s 6, 8, and 10 in FIG. 1). The magnification rating for each tissue core is the lowest magnification at which the staining intensity is definitively positive. If there is no staining at any magnification level (i.e., greater than 40×), the sample is scored negative (N). If there are multiple evaluations made on a patient, the lowest magnification (i.e., 2×<4×<10×<20×<40×) where definitely positive (PP) is obtain from the evaluations is used. There may be circumstances where if multiple evaluations are made on a patient, the alternative magnification results from the evaluations are used.

Method Three for determining a hENT1 endpoint is based on hENT1 staining pattern. The staining pattern of the hENT1 expression is rated as negative (N), segmental (S), or diffuse (D). Negative refers to no membrane staining. Segmental refers to a few areas of positive membrane staining. Diffuse refers to a more complete staining of the majority of the membranes. If there are multiple evaluations made on a patient, the maximum result (i.e., D>S>N) from the evaluations is used. There may be circumstances where if multiple evaluations are made on a patient, the minimum result from the evaluations is used.

Method Four for determining a hENT1 endpoint is based on highest percentage of hENT1 membrane staining intensity. The staining intensity for a tissue core is determined using the parameters described for the H-score (0, 1+, 2+, 3+). The percentage of each parameter represented by the tumor is determined. If a score of only 0 is obtained, the endpoint is considered Negative (N). For all other samples (e.g. those that contain scores with 0, 1+, 2+, and/or 3+), the endpoint is determined by the non-0 membrane intensity represented in the highest percentage (see FIG. 1). For example, if the sample is 50% 0, 20% 1+, 30% 2+ and 0% 3+, such sample would be referred to as 2+. If 2 intensities have the same percentage then the lower of the 2 intensities is used. A staining intensity score of 0 is referred to as Negative (N), a staining intensity score of 1+ is referred to as Weak (W), a staining intensity score of 2+ is referred to as Moderate (M), and a staining intensity score of 3+ is referred to as Strong (S). If there are multiple evaluations made on a patient, the maximum result (i.e., S>M>W) from the evaluations is used. There may be circumstances where if multiple evaluations are made on a patient, the minimum result from the evaluations is used.

Method Five for determining a hENT1 endpoint is based on the percent of tumor tissue with no staining. The percentage of negative tumor tissue represents the percentage of tumor tissue scored as 0 in the calculation of the membrane intensity. If this percentage is 100% then there is no hENT1 staining present and if it is 0% then all of the tumor tissue is staining positively for hENT1. If there are multiple evaluations made on a patient, the maximum result from the evaluations is used. There may be circumstances where if multiple evaluations are made on a patient, the minimum result from the evaluations is used.

Method Six for determining a hENT1 endpoint is based on a combination of magnification and percent of tumor tissue with no staining. If there are multiple evaluations made on a patient, the maximum combined result (magnification results 2×>4×>10×>20×>40×, combined with maximum percent of tumor tissue with no staining: 100%>50%>0%) from the evaluations is used. There may be circumstances where if multiple evaluations are made on a patient, the minimum combined result from the evaluations is used.

Additional hENT1 endpoints can be based on other combinations of hENT1 endpoints 1-6.

The outcome endpoints are calculated and correlated with hENT1 protein levels with the overall survival (OS) of patients. Overall survival is the time from start of adjuvant treatment to death.

The term “gemcitabine derivative” refers to gemcitabine that has been derivatized with a lipophilic component that facilitates transport across the plasma (and/or other) membrane(s) without the benefit of hENT1 or other nucleoside transporters. A gemcitabine derivative is typically a hydrophobic analog of gemcitabine. A gemcitabine derivative encompasses lipophilic derivatives of gemcitabine. In particular, the gemcitabine derivative can be a compound of formula I:

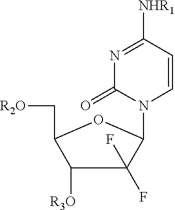

wherein R₁ and R₃ are hydrogen and R₂ is a C₁₈- or C₂₀-saturated and monounsaturated acyl group, or a pharmaceutically acceptable salt thereof. In a preferred embodiment, the gemcitabine derivative is gemcitabine-5′-elaidate. Gemcitabine-5′-elaidate ester (also referred to herein as gemcitabine-5′-elaidic acid, CP-4055, CP-4126, CO-1.01 and CO-101) has the structure of Formula (II):

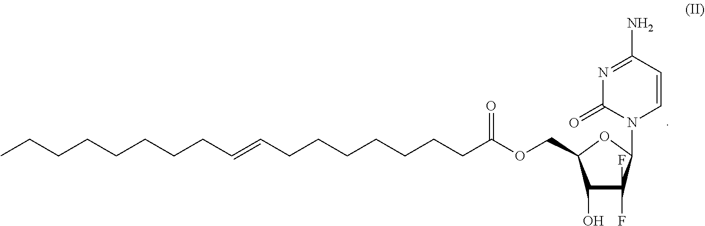

“Transport by passive diffusion” refers to transport of an agent not mediated by a specific transporter protein, e.g., hENT1. An agent that is substantially incapable of passive diffusion has a permeability across a standard cell monolayer (e.g., Caco-2 or MDCK cells or an artificial bilayer (PAMPA)) of less than 5×10⁻⁶ cm/sec, and usually less than 1×10⁻⁶ cm/sec in the absence of an efflux mechanism.

As described herein, the subject invention is directed to a method for determining whether hENT1 protein levels in a patient's pancreatic adenocarcinoma cells are below a cut-off or predetermined level so as to determine whether gemcitabine derivative therapy is appropriate. The invention is also directed to a method of treating a patient having pancreatic adenocarcinoma using a therapeutically effective amount of gemcitabine derivative when the hENT1 protein level is below the predetermined level.

“Patient” includes mammals, for example, humans. Patients include those having a disease, those suspected of having a disease, and those in which the presence of a disease is being assessed.

“Treating” or “treatment” of a disease refers to arresting or substantially slowing the growth of pancreatic adenocarcinoma cells, or at least one of the clinical symptoms of the adenocarcinoma. In certain embodiments, “treating” or “treatment” refers to arresting or reducing at least one physical parameter of the adenocarcinoma, which may or may not be discernible by the patient. In certain embodiments, “treating” or “treatment” refers to inhibiting or controlling the adenocarcinoma, either physically (e.g., stabilization of a discernible symptom), physiologically (e.g., stabilization of a physical parameter), or both.

“Therapeutically effective amount” refers to the amount of a compound that, when administered to a subject for treating pancreatic adenocarcinoma, is sufficient to affect such treatment of the adenocarcinoma. The “therapeutically effective amount” may vary depending, for example, on the gemcitabine derivative selected, the stage of the adenocarcinoma, the age, weight and/or health of the patient and the judgment of the prescribing physician. An appropriate amount in any given instance may be readily ascertained by those skilled in the art or capable of determination by routine experimentation.

A “sample” or “biological sample” is a biological specimen containing genomic DNA, RNA (including mRNA), protein, or combinations thereof, obtained from a subject. Examples include, but are not limited to, chromosomal preparations, peripheral blood, urine, saliva, tissue biopsy, surgical specimen, bone marrow, amniocentesis samples and autopsy material. In one example, a sample includes genomic DNA or RNA. In some examples, the sample is a cytogenetic preparation, for example which can be placed on microscope slides. In particular examples, samples are used directly, or can be manipulated prior to use, for example, by fixing (e.g., using formalin).

Methods described herein are related to a variety of cancers. In some instances, cancer can be a metastatic cancer. Examples of cancers related to the methods described herein include, but are not limited to, sarcoma, malignant melanoma, prostate cancer, breast cancer, pancreatic cancer, colon cancer (such as a colon carcinoma), glioma, leukemia, liver cancer, colon cancer (including small intestine cancer), breast cancer, pancreatic cancer, melanoma (e.g., metastatic malignant melanoma), acute myeloid leukemia, kidney cancer, bladder cancer, ovarian cancer, prostate cancer, renal cancer (e.g., renal cell carcinoma), glioblastoma, brain tumors, chronic or acute leukemias including acute lymphocytic leukemia (ALL), adult T-cell leukemia (T-ALL), chronic myeloid leukemia, acute lymphoblastic leukemia, chronic lymphocytic leukemia, lymphomas (e.g., Hodgkin's and non-Hodgkin's lymphoma, lymphocytic lymphoma, primary CNS lymphoma, T-cell lymphoma, Burkitt's lymphoma, anaplastic large-cell lymphomas (ALCL), cutaneous T-cell lymphomas, nodular small cleaved-cell lymphomas, peripheral T-cell lymphomas, Lennert's lymphomas, immunoblastic lymphomas, T-cell leukemia/lymphomas (ATLL), entroblastic/centrocytic (cb/cc) follicular lymphomas cancers, diffuse large cell lymphomas of B lineage, angioimmunoblastic lymphadenopathy (AILD)-like T cell lymphoma and HIV associated body cavity based lymphomas), embryonal carcinomas, undifferentiated carcinomas of the rhino-pharynx (e.g., Schmincke's tumor), Castleman's disease, Kaposi's Sarcoma, multiple myeloma, Waldenstrom's macroglobulinemia and other B-cell lymphomas, nasopharangeal carcinomas, bone cancer, skin cancer, cancer of the head or neck, cutaneous or intraocular malignant melanoma, uterine cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), tumor angiogenesis, spinal axis tumor, brain stem glioma, pituitary adenoma, epidermoid cancer, squamous cell cancer, or environmentally induced cancers including those induced by asbestos, e.g., mesothelioma. In another embodiment, methods described herein can be useful for treating a combination of two or more types of cancer. In some aspects the methods are useful to treat individual patients diagnosed with cancer.

This invention is useful in any cancer types wherein decreased hENT1 protein expression limits the effectiveness of gemcitabine, including pancreatic adenocarcinoma, and the like.

Generally, it is expected that treatment with gemcitabine derivative will be utilized in patients that have undergone resection of the adenocarcinoma or other cancer. However, an alternative embodiment provides for treatment without resection to reduce structural damage to the organ in which the cancer has arisen.

The subject invention is also directed to kits for determination of whether a gemcitabine derivative is suitable in the treatment of a pancreatic adenocarcinoma of a patient. The kit typically includes an antibody to hENT1 protein suitable for staining a tissue section of the patient's adenocarcinoma cells, and instructions for use of the antibody in staining the tissue section. The kit permits the determination of whether the hENT1 level is below a predetermined level, thereby informing the practitioner whether treatment of the pancreatic adenocarcinoma with the gemcitabine derivative is appropriate.

The invention having now been described by way of written description, those of skill in the art will recognize that the invention can be practiced in a variety of aspects and that the foregoing description and examples below are for purposes of illustration and not limitation of the claims that follow.

### EXAMPLES

While alternative aspects have been shown and described herein, it will be obvious to those skilled in the art that such aspects are provided by way of example only. Numerous variations, changes, and substitutions will occur to those skilled in the art without departing from the invention. It should be understood that various alternatives to the aspects of the invention described herein can be employed in practicing the invention. It is intended that the following claims define the scope of the invention and that methods and structures within the scope of these claims and their equivalents be covered thereby.

### Example 1

### A Retrospective Study to Evaluate Tumor hENT1 Expression and its Relationship to Treatment Outcome in Patients with Pancreatic Cancer Who Participated in The RTOG 9704 Study

Study Design

A retrospective, observational study of hENT1 expression in pancreatic adenocarcinoma patients that participated in the Radiation Therapy Oncology Group (RTOG) led trial designated RTOG 9704 study has been conducted and initial data is presented here. Tumor tissue samples are available from patients that participated in the RTOG 9704 trial: Fluorouracil-based Chemoradiation with Either Gemcitabine or Fluorouracil Chemotherapy after Resection of Pancreatic Adenocarcinoma. The RTOG 9704 study and its results are further described in Regine, W. F. et al. (2008) JAMA 299(9):1019-1026; Regine, W. F. et al. (2011) Ann. Surg. Oncol. 18:1319-1326.

The data from RTOG 9704 has been used to establish a hENT1 cut-off that identifies patients who will benefit from gemcitabine. Patients having hENT1 levels below the hENT1 cut-off will benefit from treatment with a gemcitabine derivative, particularly gemcitabine-5′-elaidate. In addition, the patients not treated with gemcitabine from the RTOG 9704 study will be used to evaluate the prognostic value of hENT1 expression.

Formalin-fixed, paraffin-embedded (FFPE) specimens have been retrieved from tissue repositories. The specimens were obtained from patients prior to adjuvant therapy. Tissue was processed into tissue microarrays (TMA) at the RTOG Biospecimen Resource center and IHC staining was performed in a central laboratory (Ventana Medical Systems). Stained slides were interpreted by pathologists blind to study outcomes.

Clinical information about the patient, the pancreatic cancer and treatment were gathered to the extent possible and correlated with levels of hENT1 expression found in tumor samples. Minimum clinical information comprises patient age, date and disease stage at diagnosis, date of starting adjuvant therapy and overall survival (OS).

The objectives of this study include: 1. to define a cut-off level of hENT1 protein level that is predictive of an OS benefit of adjuvant gemcitabine, 2. to estimate the distribution of hENT1 protein levels in pancreatic tumors, 3. to investigate the association between hENT1 cut-off levels previously defined in the literature and OS in patients treated with adjuvant gemcitabine, 4. to evaluate whether hENT1 protein levels are prognostic indicator of OS in patients treated with non-gemcitabine therapy, and 5. to establish a hENT1 protein level at or below which a patient will benefit from treatment with a gemcitabine derivative, such as gemcitabine-5′-elaidate.

The tissues used in this study are from the Radiation Therapy Oncology Group (RTOG) Biospecimen Resource and the tumor tissues are from pancreatic adenocarcinoma from patients who have undergone a tumor resection.

Data Collection

The clinical data collected via case report forms in the RTOG 9704 study were electronically transferred from the existing databases. The data includes, but is not be limited to, the following data.

1. Demographics:
Date of birth
Gender (female, male)
WHO Performance status (0-4)
Country.

2. Tumor History:
Date of initial diagnosis or pathology report
TNM tumor stage.

3. Treatment and Outcome:
Date of adjuvant therapy initiation
Date of death
Date of disease progression (tumor recurrence or metastatic disease)
Dose and duration of adjuvant therapy.

Tissue Micro Arrays

Samples have been centralized within the RTOG Biospecimen Resource facility. Tissue micro arrays (TMA) were also constructed in the RTOG Biospecimen Resource facility. Three cores from each tumor specimen were arrayed on three separate TMAs. A total of 220 cases were arrayed on the TMAs.

Tissue Sample Management and Analysis

Slides from TMAs were prepared and forwarded to the core pathology laboratory (Ventana Medical Systems), and stained by SP120 rabbit monoclonal antibody for detection of hENT1 protein according to established methods. The following parameters were recorded for each specimen based on scoring by a pathologist blinded to the patient's clinical characteristics and outcome:
Intensity of staining (e.g. 0=no stain, 1+=weakly positive, 2+=moderately positive, 3+=strongly positive)
Percentage of tumor cells with staining at each intensity level (% of tumor tissue specifically excluding non-malignant cells/regions)
Pattern of staining (segmented or diffuse)
Cellular compartment (membrane or cytoplasm).

Cells from the islets of Langerhans or infiltrating lymphocytes were used, where present, as the internal positive controls between normal and tumor tissue, as well as for the staining procedure. In other embodiments controls can include different cell lines representing different staining parameters, such as ASPC1 cells that will demonstrate no staining, HeLa cells which will stain moderately, and PANC1 cells that stain strongly positive.

The results of the pathologist scoring were collected in a database. FIGS. 1 and 2 are examples of scoring sheets from such a database.

Statistical Considerations

1. Sample size.

The sample sizes for the tissue samples from the RTOG study used to generate this initial data are 194 samples from the RTOG-9704 study with 100 samples from gemcitabine-treated patients and 94 samples from patients treated with 5-Fluorouracil (5FU)-based chemotherapy and 5FU chemotherapy following resection).

The primary objective of this study is to establish a cut-off in hENT1 expression that is predictive of a therapeutic response to treatment with gemcitabine.

In order to define the hENT1 cut-off, it is not necessary to demonstrate a statistically significant difference between gemcitabine and 5FU within each hENT1 subgroup. The most important aspect to defining the hENT1 cut-off is observing a differential treatment effect between the hENT1 low and high subgroups. The following power and sample size calculations for the comparison of gemcitabine versus 5FU are provided to support the analysis and interpretation of the study results and ultimately define a cut-off in hENT1 expression.

For comparisons of OS between gemcitabine and 5FU within a subgroup of patients defined by hENT1 (High v. Low), the following table provides the power to detect a significant difference for various sample sizes and treatment effects.

TABLE 1
Power Detection

| Hazard ratio | No. of | | No. of | |
|---|---|---|---|---|
| (gem v. 5FU) | death events | Power | death events | Power |
| 0.5 | 70 | 80% | 90 | 90% |
| 0.6 | 130 | 80% | 170 | 90% |

Assumes a two-sided significance level of 0.05.

### Example 2

### hENT1 Endpoint as Determined by Combination Algorithm of Magnification And Percent Staining

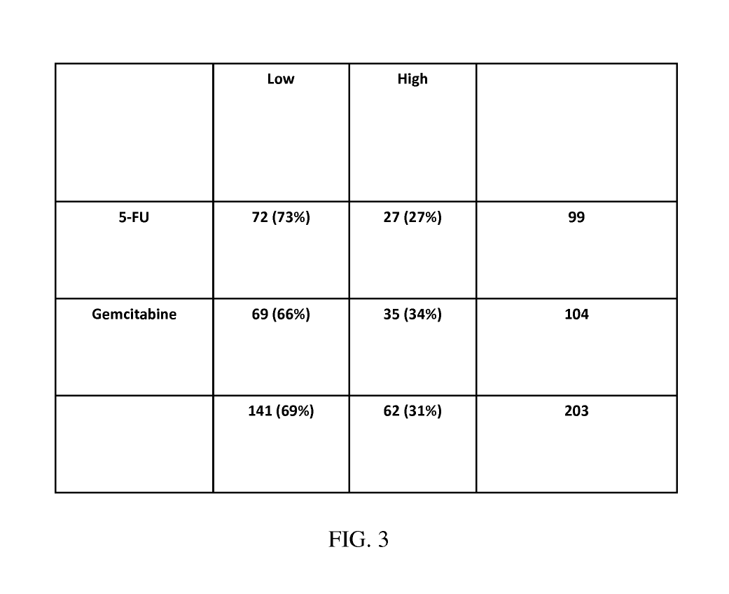

A hENT1 endpoint was calculated and used to correlate hENT1 expression with the overall survival (OS) of patients from the RTOG 9704 study described in Example 1. Results obtained from the RTOG 9704 study and analyzed for hENT1 endpoint by Method Six are set forth in FIGS. 3-8.

FIG. 3 shows the frequency of patients across the 2 subgroups of hENT1 expression defined categorically as High and Low, where a sample is defined as High when the sample has the criterion of at least 50% of the tumor displays hENT1 membrane staining with use of a 10× ocular using a light microscope (100× total magnification) and a sample is defined as Low when the sample has the criterion of less than 50% of the tumor tissue displays hENT1 membrane staining with use of a 10× ocular using a light microscope (100× total magnification). If multiple samples were obtained from a patient, if any of the samples were classified as High, then that patient is classified as High.

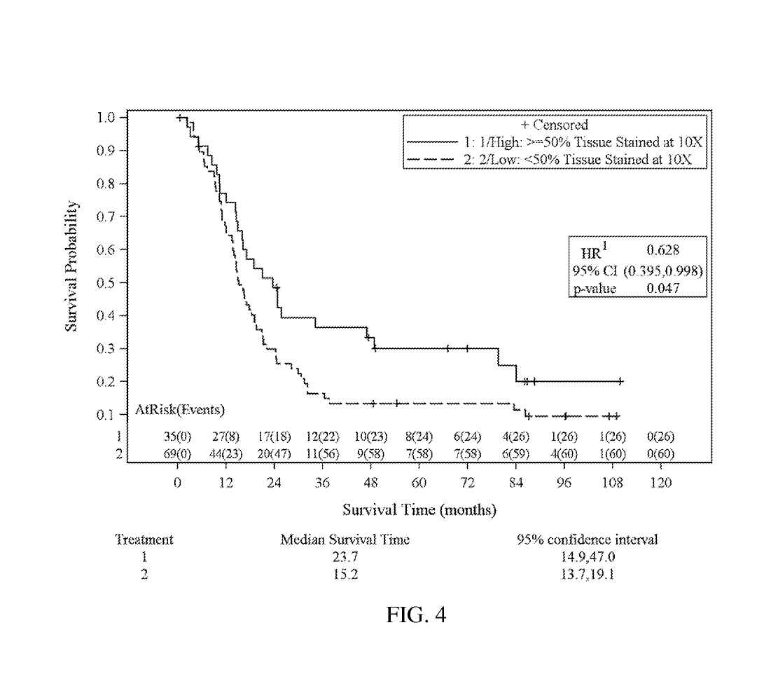

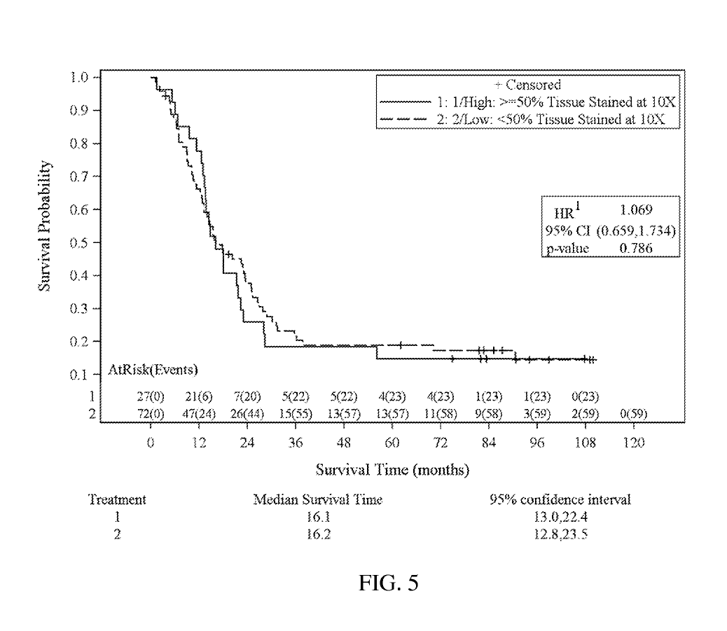

FIG. 4 illustrates that in gemcitabine-treated patients, the subgroup with Low hENT1 expression exhibited shorter OS as compared to the subgroup that exhibited High hENT1 expression. In contrast, FIG. 5 shows that 5FU treated patients do not demonstrate improvement in OS between the High and Low subgroups of patients.

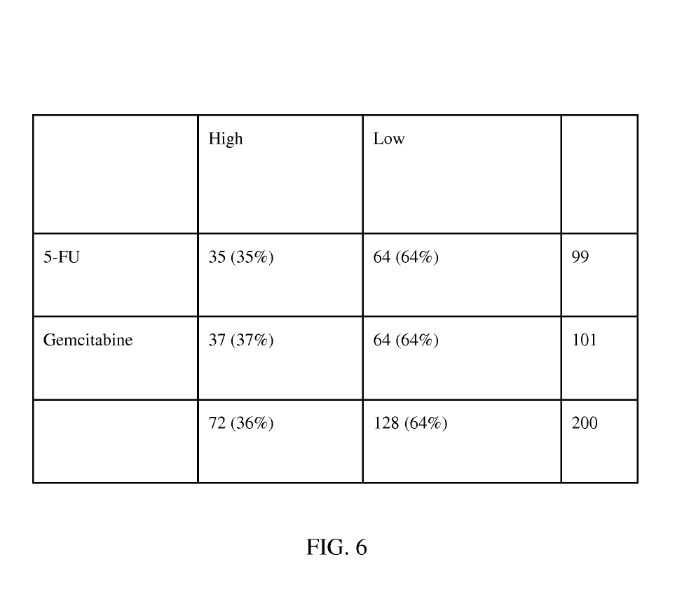

FIG. 6 shows the frequency of patients across the 2 subgroups of hENT1 expression defined categorically as High and Low, where a sample is defined as High when the sample is determined to have:
a) definitely positive (PP) tumor tissue observed at a magnification of 2×, 4×, and/or 10×, AND
b) the percent of negative tumor tissue (Membrane intensity classified as 0) is <=50%.

A sample is defined as Low when the sample is determined to have either:
a) >50% of negative tumor tissue (Membrane intensity is 0) OR
b) definitely positive (PP) tumor tissue observable only at a magnification level of 20× or 40× or not at any of the tested magnifications.

If multiple samples were obtained from a patient, if any of the samples were classified as High, then that patient is classified as High.

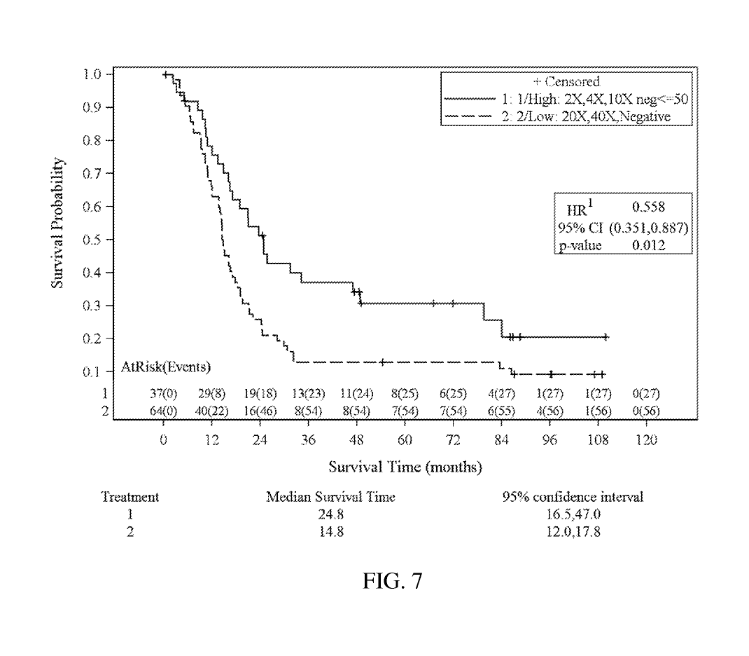

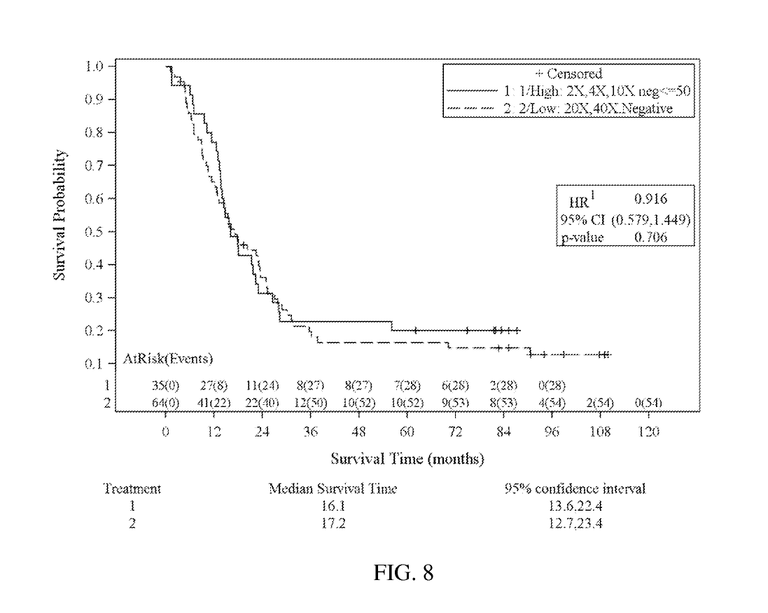

FIG. 7 illustrates that in gemcitabine-treated patients, the subgroup with Low hENT1 expression exhibited shorter OS as compared to the subgroup that exhibited High hENT1 expression. In contrast, FIG. 8 shows that 5FU treated patients do not demonstrate improvement in OS between the High and Low subgroups of patients.

### Example 3

### Characterization of hENT1 in Matched Primary and Metastatic Pancreatic Adenocarcinomas

A study was conducted to determine whether the expression of hENT1 is the same between primary pancreatic adenocarcinoma and metastatic lesions. The study evaluated hENT-1 by IHC in matched pancreatic ductal adenocarcinoma in the primary tumor and in lymph node metastases. The study is based upon paraffin embedded pancreatic cancer specimens from patients operated upon and resected with radical attempt. Sixteen matched primary and metastatic pancreatic adenocarcinomas were obtained from Lund University (Department of Surgery, Clinical Sciences Lund, Lund University, Lund, Sweden). From the identified paraffin embedded specimens, sections were taken for measuring hENT-1 expression for the primary and metastatic pancreatic cancer.

Slides from tumor tissue blocks were prepared and forwarded to the core pathology laboratory (Ventana Medical Systems), and stained by SP120 rabbit monoclonal antibody for detection of hENT1 protein according to established methods. The following parameters were recorded for each specimen based on scoring by a pathologist blinded to the patient's clinical characteristics and outcome:
Intensity of staining (e.g. 0=no stain, 1+=weakly positive, 2+=moderately positive, 3+=strongly positive)
Percentage of tumor cells with staining at each intensity level (% of tumor tissue specifically excluding non-malignant cells/regions)
definitely positive (PP) tumor tissue observed at a magnification of 2×, 4×, 10×, 20×, and 40×

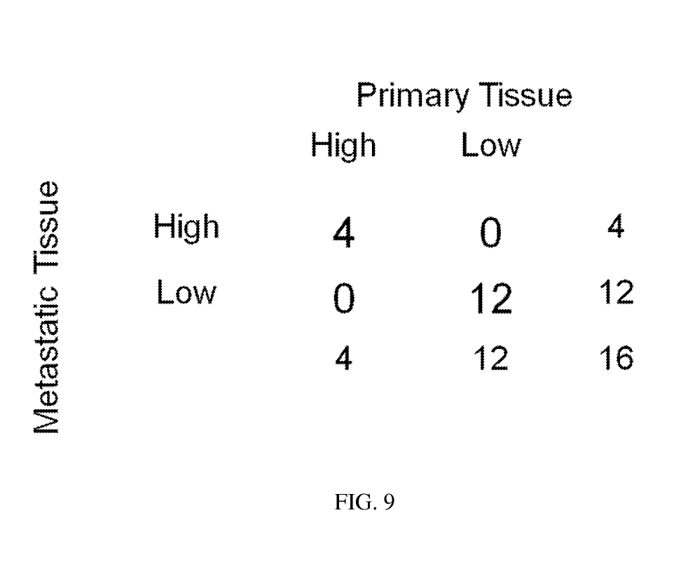

An H-score was derived for hENT1 in both the primary and matched metastatic lesion. When a linear regression analysis was performed the correlation of H-scores from the 16 matched samples was 0.78. When the pre-specified algorithm (high hENT1=resolvable membrane hENT1 staining in greater than 50% of tumor using the 10× objective) was employed to define hENT1 status the concordance was 100% between the primary and metastatic samples (FIG. 9). These results indicate that hENT1 expression is similar between the primary pancreatic ductal adenocarcinoma and metastatic lesions from the local lymph nodes of the same patient.

### Example 4

### A Retrospective Study to Evaluate Tumor Human Equilibrative Nucleoside Transporter 1 (hENT1) Expression and its Relationship to Treatment Outcome in Patients with Pancreatic Cancer Who have Received Gemcitabine

A study was conducted to determine the prevalence and predictive value of hENT1 expression in pancreatic cancer patients undergoing gemcitabine therapy. The study evaluated hENT-1 by IHC in pancreatic ductal adenocarcinoma in the primary tumor and in distant metastases. The study is based upon paraffin embedded pancreatic cancer specimens obtained from a resection or biopsy.

Slides from tumor tissue blocks were prepared and forwarded to the core pathology laboratory (Ventana Medical Systems), and stained by SP120 rabbit monoclonal antibody for detection of hENT1 protein according to established methods. A total of 204 tissue specimens (134 primary and 70 metastatic) from pancreatic adenocarcinomas were obtained from Virginia Mason University. In the primary tumor set, 90/134 patients received gemcitabine therapy and in the metastatic tumor set 63/70 patients received gemcitabine therapy. The percentages of tumor specimens scored as hENT1 low were 61% and 77%, respectively, in the primary and metastatic tumor sets. The samples were categorized as hENT1 High or Low using the Method Six algorithm described in Example 2.

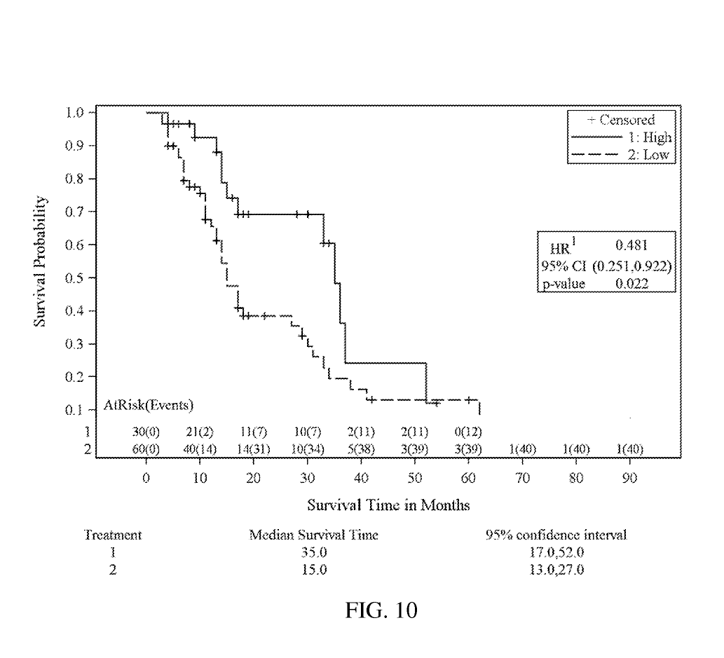

FIG. 10 is a Kaplan-Meier plot of OS for patients treated with gemcitabine and hENT1 status (High [1] and Low [2]) based on primary tumor tissue. The hENT1 expression was determined using the Method Six algorithm described in Example 2.

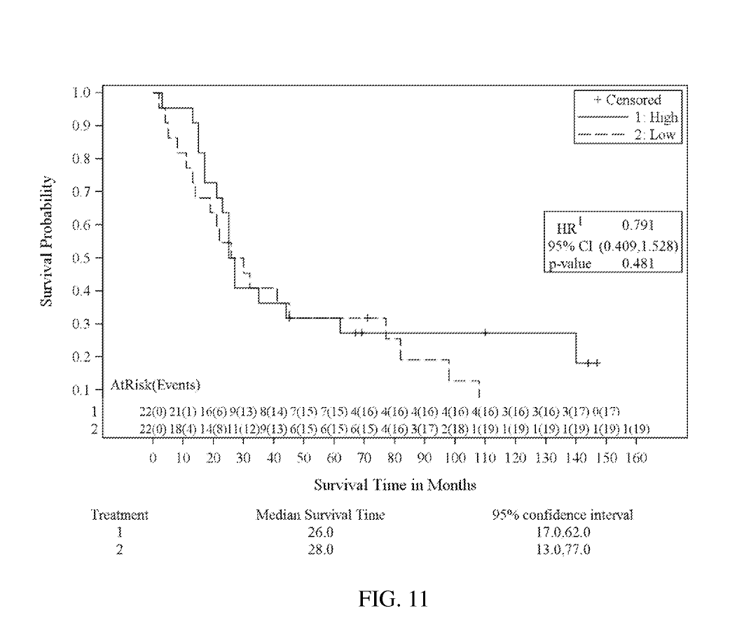

FIG. 11 is a Kaplan-Meier plot of OS for patients never treated with gemcitabine and hENT1 status (High [1] and Low [2]) based on primary tumor tissue. The hENT1 expression was determined using the Method Six algorithm described in Example 2.

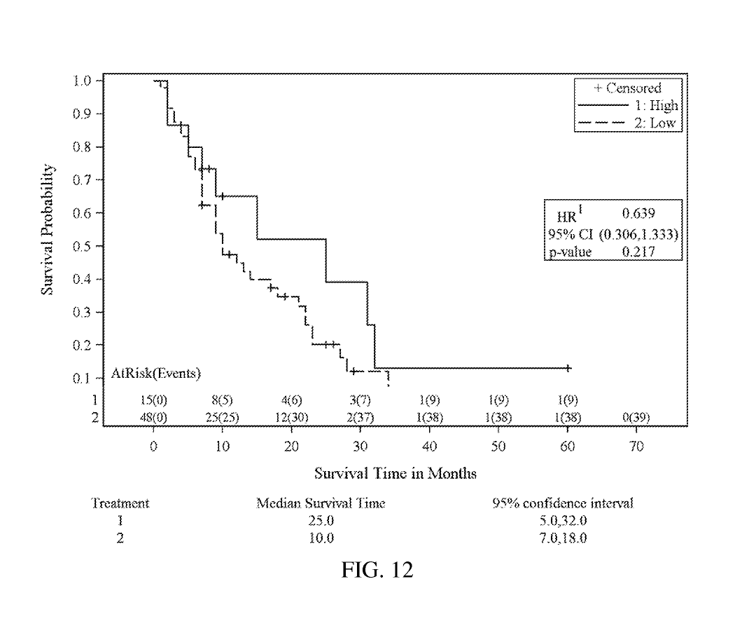

FIG. 12 is a Kaplan-Meier plot of OS for patients treated with gemcitabine and hENT1 status (High [1] and Low [2]) based on metastatic tumor tissue. The hENT1 expression was determined using the Method Six algorithm described in Example 2.

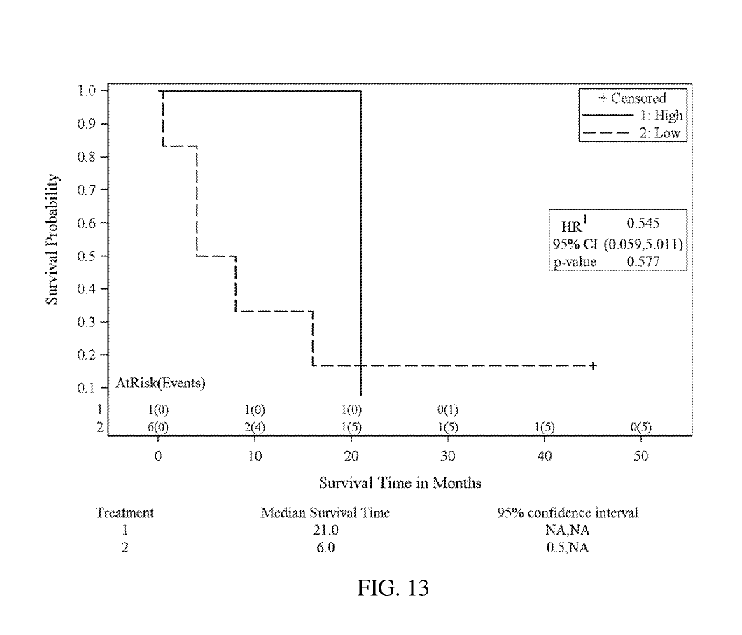

FIG. 13 is a Kaplan-Meier plot of OS for patients never treated with gemcitabine and hENT1 status (High [1] and Low [2]) based on metastatic tumor tissue. The hENT1 expression was determined using the Method Six algorithm described in Example 2.

### Example 5

### hENT1 Endpoint as Determined by Magnification Algorithm

A hENT1 endpoint was calculated and used to correlate hENT1 expression with the overall survival (OS) of patients from the RTOG 9704 study described in Example 1.

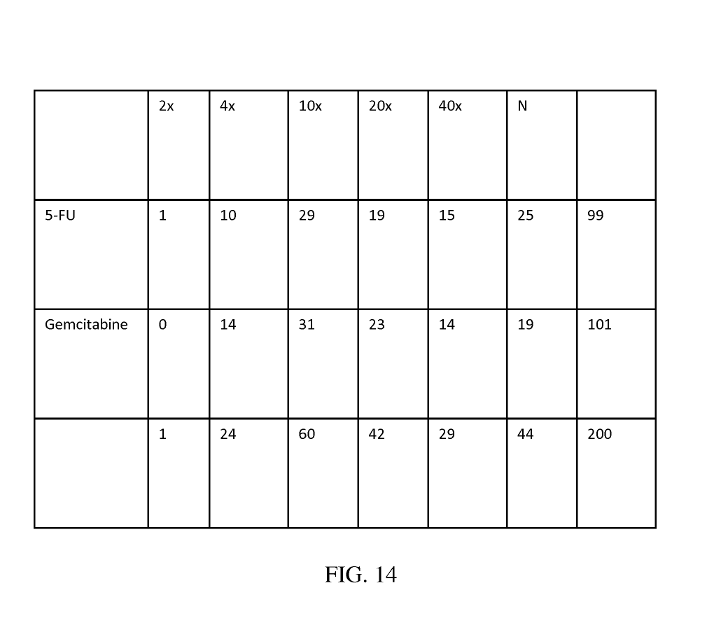

Preliminary results obtained from the RTOG 9704 study and analyzed for hENT1 endpoint by Method Two are set forth in FIGS. 14-16.

FIG. 14 shows the frequency of patients across the 6 subgroups of hENT1 expression defined categorically as Negative (N), 2×, 4×, 10×, 20×, and 40×.

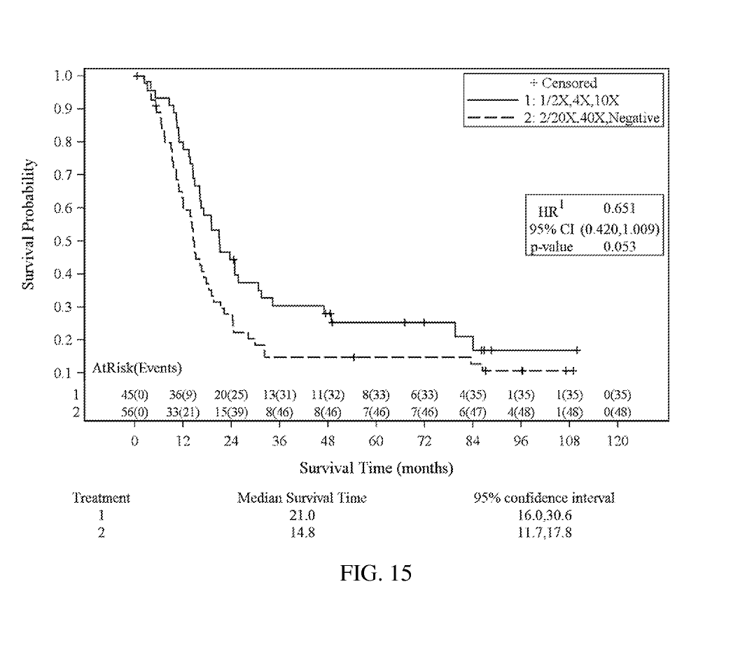

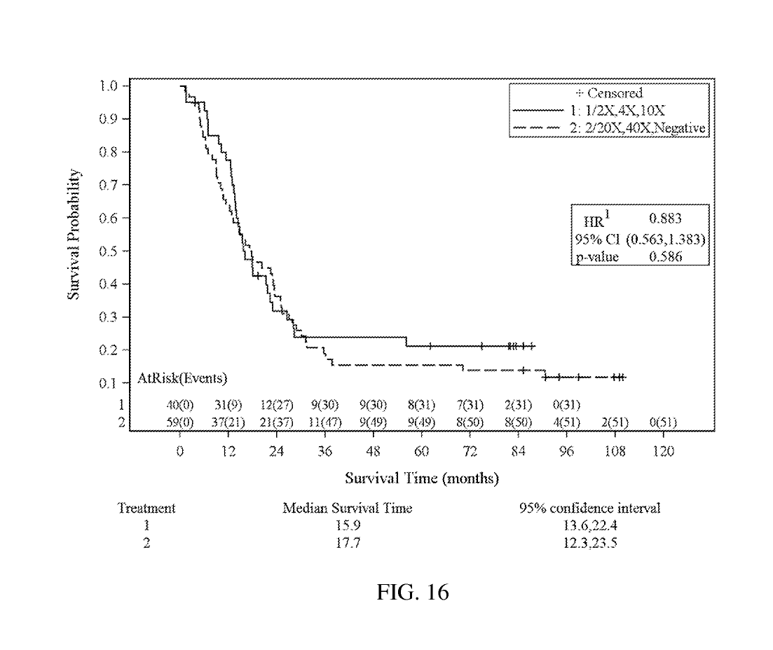

FIG. 15 illustrates that in gemcitabine-treated patients, the subgroup with negative hENT1 expression or definitely positive (PP) hENT1 expression only magnification at 20× or 40× exhibited shorter OS as compared to the subgroup that exhibited definitely positive (PP) hENT1 expression at magnifications of 2×, 4×, or 10×. In contrast, FIG. 16 shows that 5FU treated patients do not demonstrate an improvement in OS for the subgroup of patients with clearly positive hENT1 expression at magnifications of 2×, 4×, or 10×.

### Example 6

### hENT1 Endpoint as Determined by Algorithm of Percent Negative Staining

A hENT1 endpoint was calculated and used to correlate hENT1 expression with the overall survival (OS) of patients from the RTOG 9704 study described in Example 1.

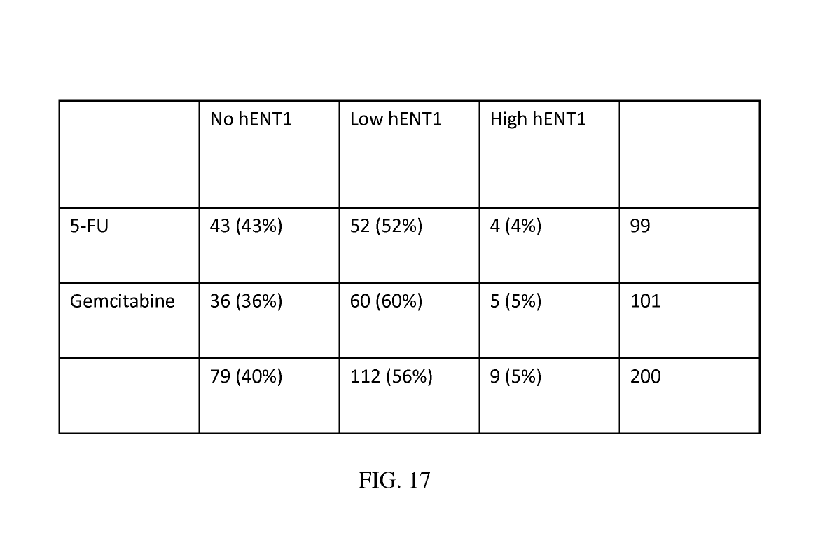

Preliminary results obtained from the RTOG 9704 study and analyzed for hENT1 endpoint by Method Five are set forth in FIGS. 17-20.

FIG. 17 shows the frequency of patients across three subgroups of hENT1 expression. A No hENT1 sample is defined as a sample wherein the membrane intensity of 0 is greater than 50 percent (see FIG. 1). A High hENT1 sample is defined as a sample wherein the membrane intensity H-score of 2+(% Moderate) plus 3+(% Strong) is greater than 50 percent. Any sample that is not either a No hENT1 sample or a High hENT1 sample is defined as a Low hENT1 sample.

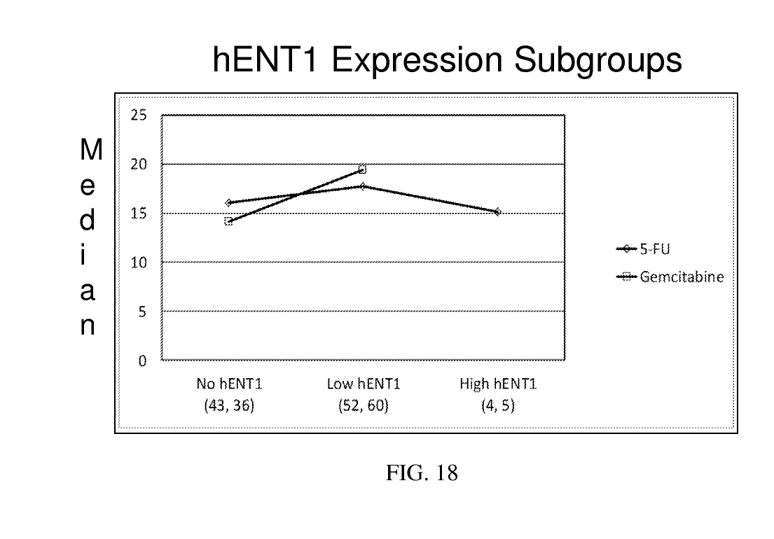

FIG. 18 illustrates that as hENT1 expression increases from No to Low to High, the median OS for gemcitabine-treated patients increases. In contrast, 5FU treated patients do not demonstrate an improvement in OS with increasing hENT1 expression.

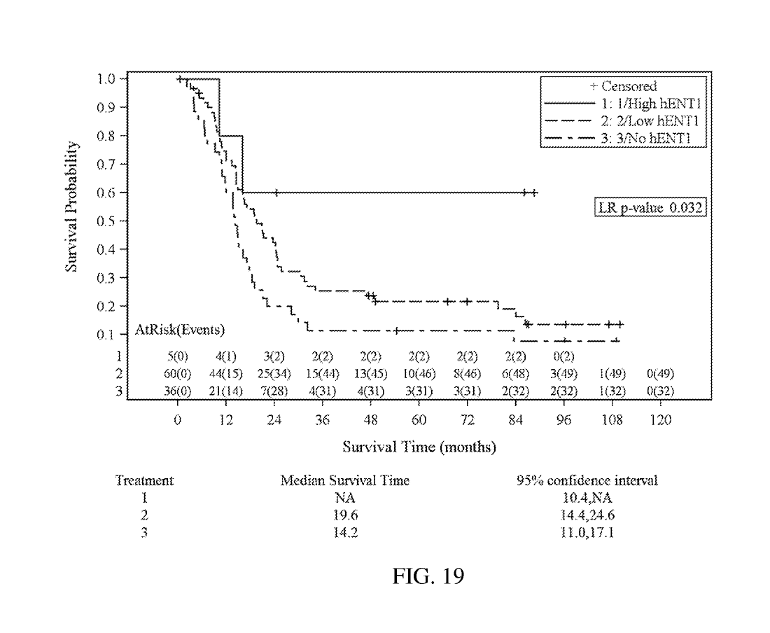

FIG. 19 illustrates that in gemcitabine-treated patients, the subgroup defined as No hENT1 exhibited shorter OS as compared to the subgroups with some detectable hENT1 expression (Low and High hENT1 expression).

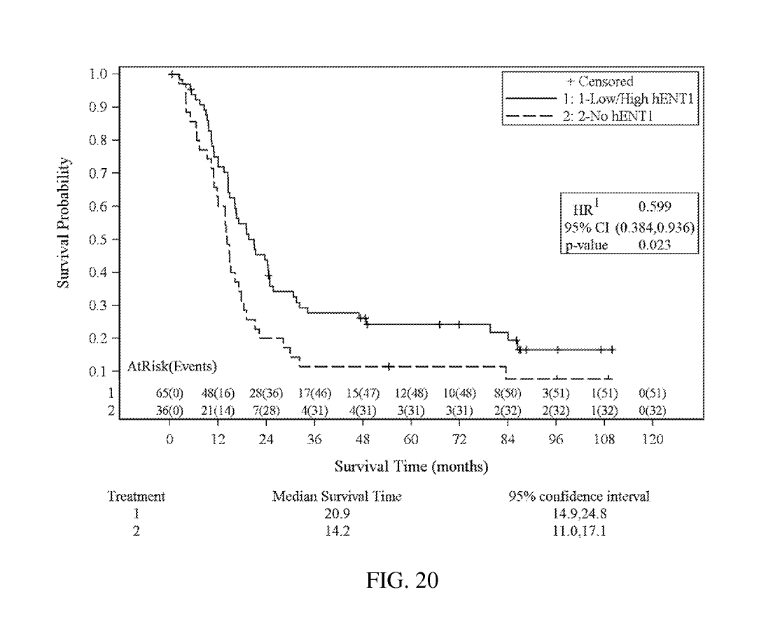

FIG. 20 combines the Low and High hENT1 subgroups and compares the combined subgroup to the No hENT1 expression subgroups and again shows that patients with observable hENT1 expression and treated with gemcitabine exhibit longer OS than patients with no hENT1 expression.

### Example 7

### hENT1 Endpoint as Determined by H-Score Algorithm

A hENT1 endpoint was calculated and used to correlate hENT1 expression with the overall survival (OS) of patients from the RTOG 9704 study described in example 1.

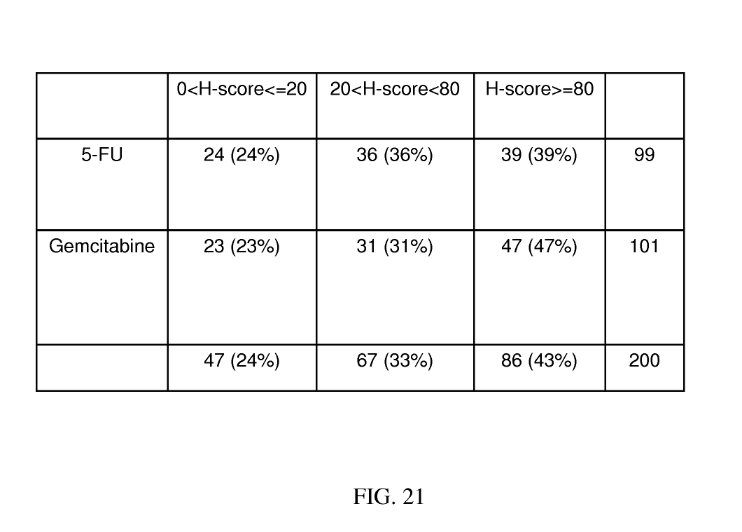

Preliminary results obtained from the RTOG 9704 study and analyzed for hENT1 endpoint by Method One are set forth in FIGS. 21-24.

FIG. 21 shows the frequency of patients across three approximately equal sized subgroups of increasing hENT1 expression.

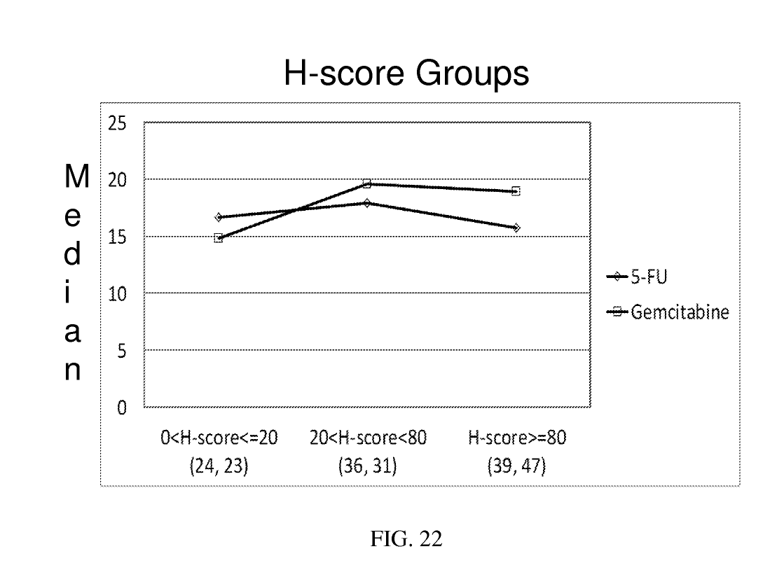

FIG. 22 illustrates that as hENT1 expression increases, the median OS for gemcitabine-treated patients increases. In contrast, 5FU treated patients do not demonstrate an improvement in OS with increasing hENT1 expression.

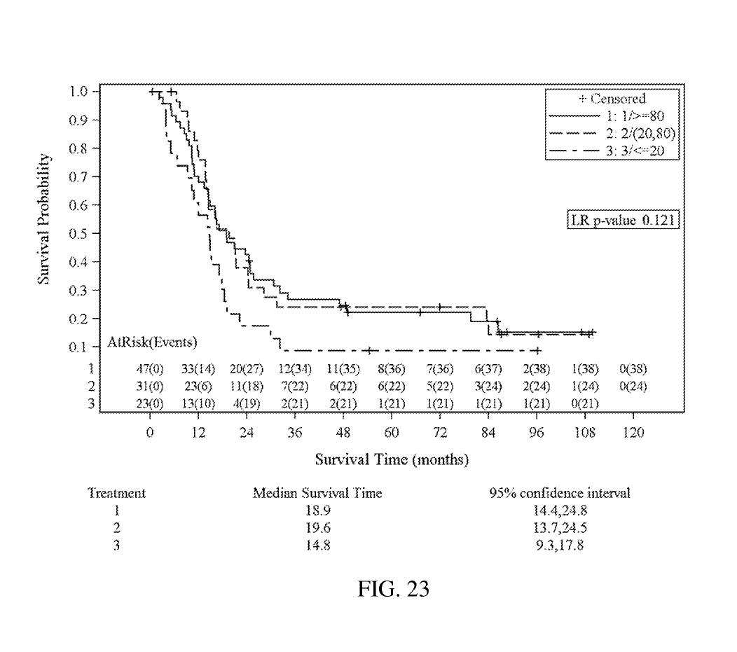

FIG. 23 illustrates that in gemcitabine-treated patients, the subgroup with low hENT1 expression (H-score<=20) exhibited shorter OS as compared to the subgroups with some detectable hENT1 expression (H-score between 20 and 80 and H-score>=80).

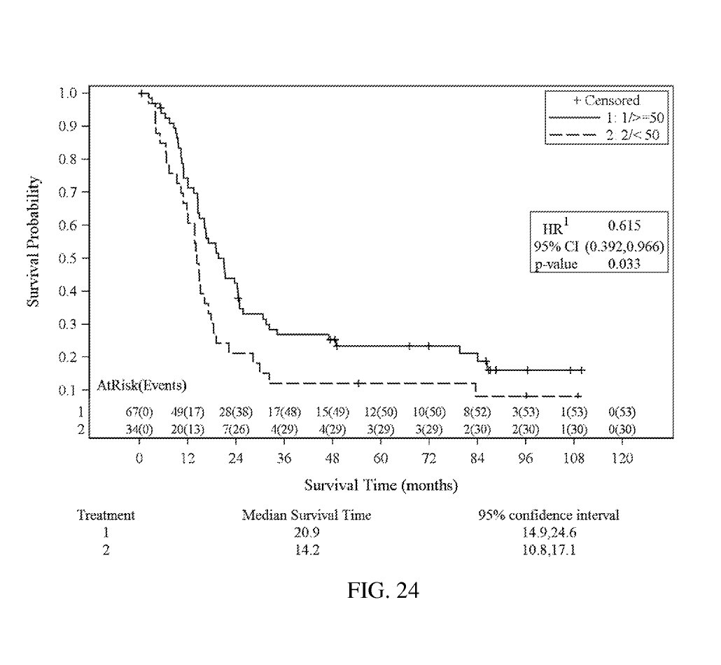

FIG. 24 shows that patients treated with gemcitabine with an H-score>=50 exhibited longer OS than patients with an H-score <50.

### Example 8

### hENT1 Endpoint as Determined by Minimum Staining Intensity Algorithm

A hENT1 endpoint was calculated and used to correlate hENT1 expression with the overall survival (OS) of patients from the RTOG 9704 study as described in example 1.

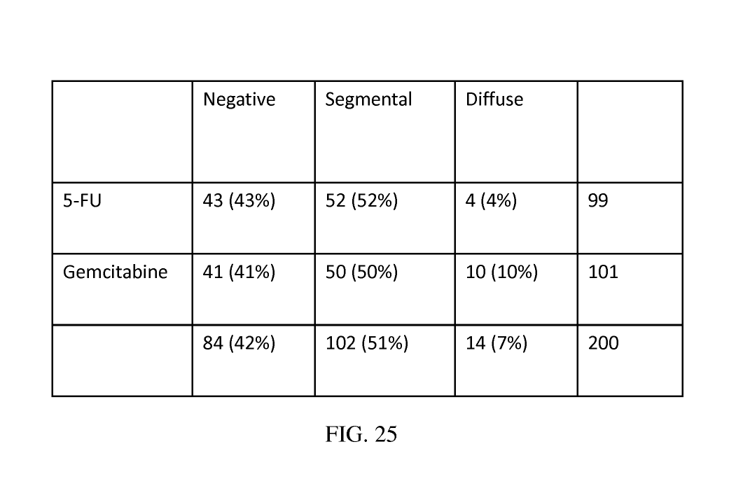

Preliminary results obtained from the RTOG 9704 study and analyzed for hENT1 endpoint by Method Three are set forth in FIGS. 25-28.

FIG. 25 shows the frequency of patients across the three subgroups of hENT1 expression defined categorically as Negative (N), Segmental (S), and Diffuse (D).

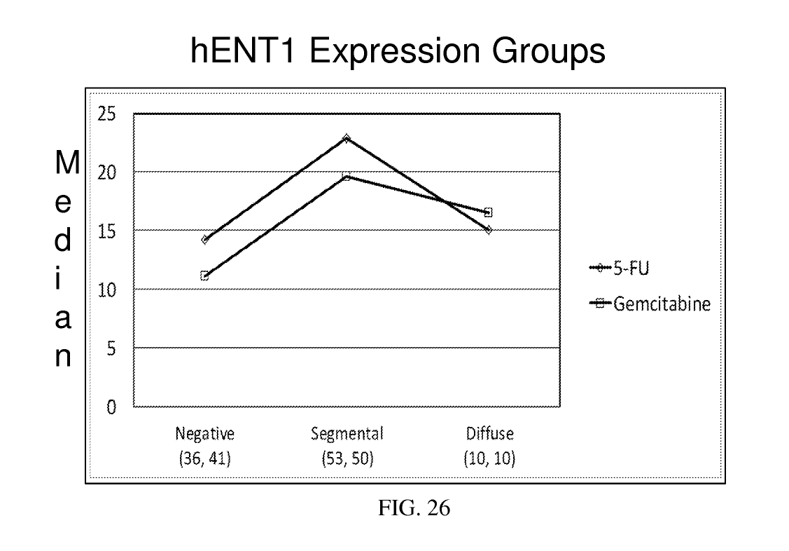

FIG. 26 illustrates that as hENT1 expression increases from Negative to Segmental to Diffuse, the median OS for gemcitabine-treated patients increases. In contrast, 5FU treated patients do not demonstrate an improvement in OS with increasing hENT1 expression.

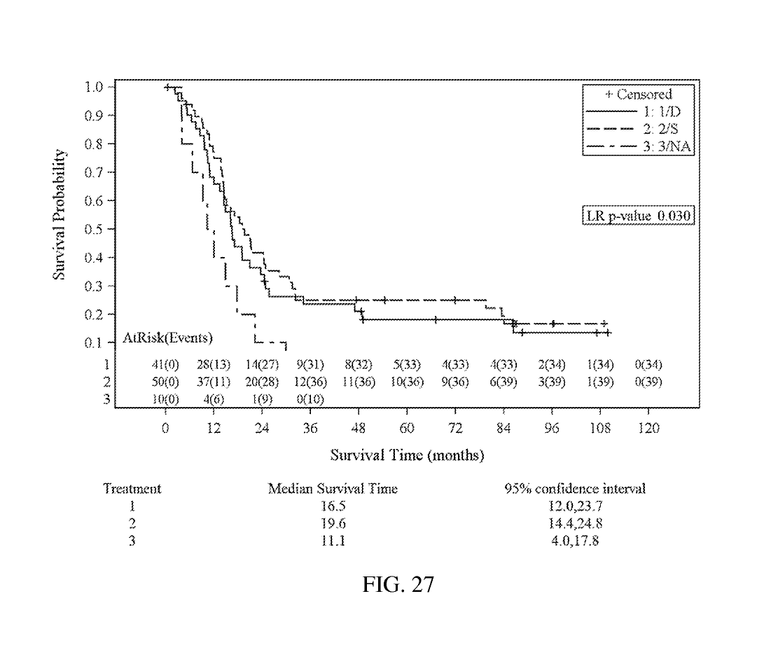

FIG. 27 illustrates that in gemcitabine-treated patients, the subgroup with negative hENT1 expression exhibited shorter OS as compared to the subgroups with some detectable hENT1 expression (Segmental or Diffuse).

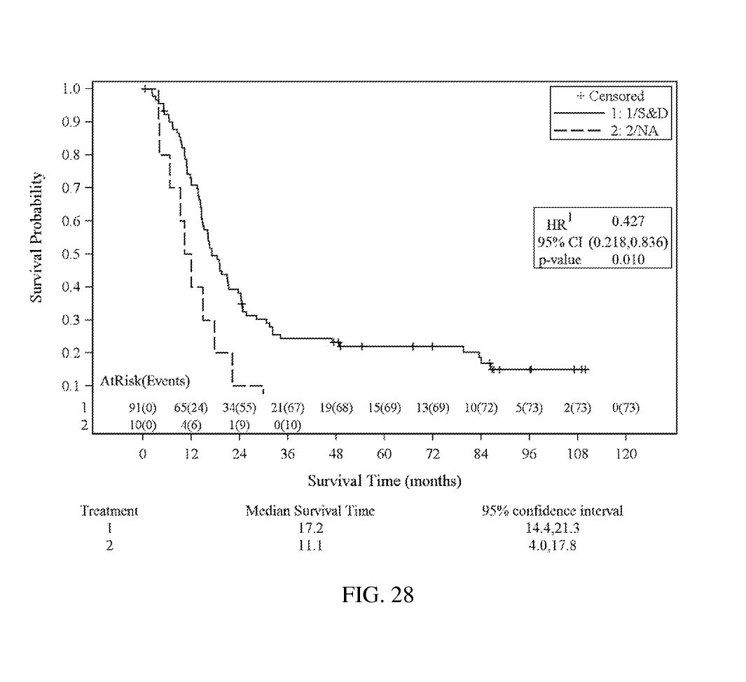

FIG. 28 shows that patients treated with gemcitabine with detectable hENT1 expression (S or D) exhibit longer OS than patients with no hENT1 expression.

### Example 9

### hENT1 Endpoint as Determined by Maximum Staining Intensity Algorithm

A hENT1 endpoint was calculated and used to correlate hENT1 expression with the overall survival (OS) of patients from the RTOG 9704 study.

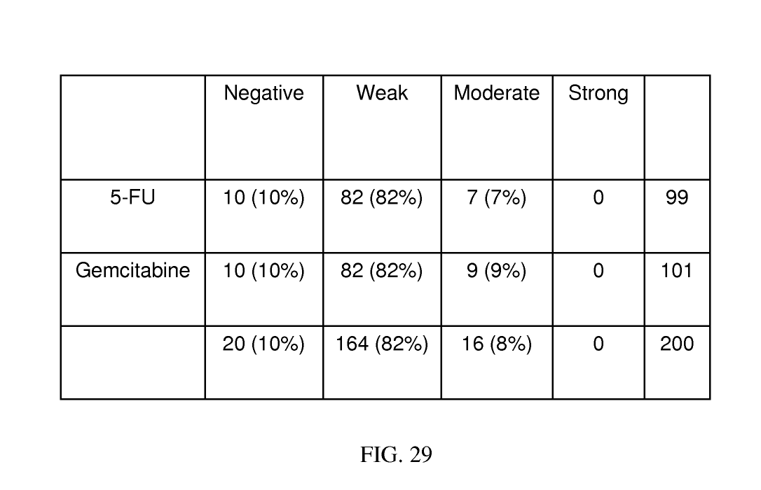

Preliminary results obtained from the RTOG 9704 study and analyzed for hENT1 endpoint by Method Four are set forth in FIGS. 29-31.

FIG. 29 shows the frequency of patients across the four subgroups of hENT1 expression defined categorically as Negative (N), Weak (W), Moderate (M) and Strong (S).

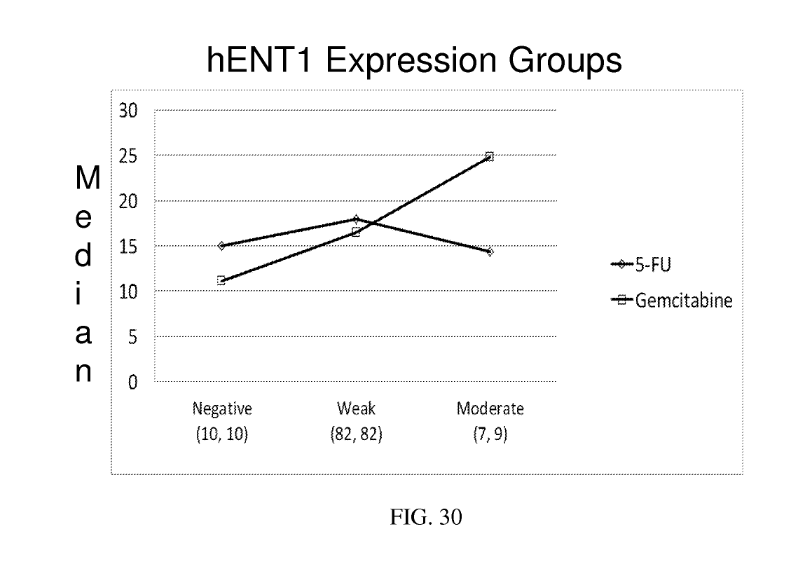

FIG. 30 illustrates that as hENT1 expression increases from Negative to Weak to Moderate to Strong, the median OS for gemcitabine-treated patients increases. In contrast, 5FU treated patients do not demonstrate an improvement in OS with increasing hENT1 expression.

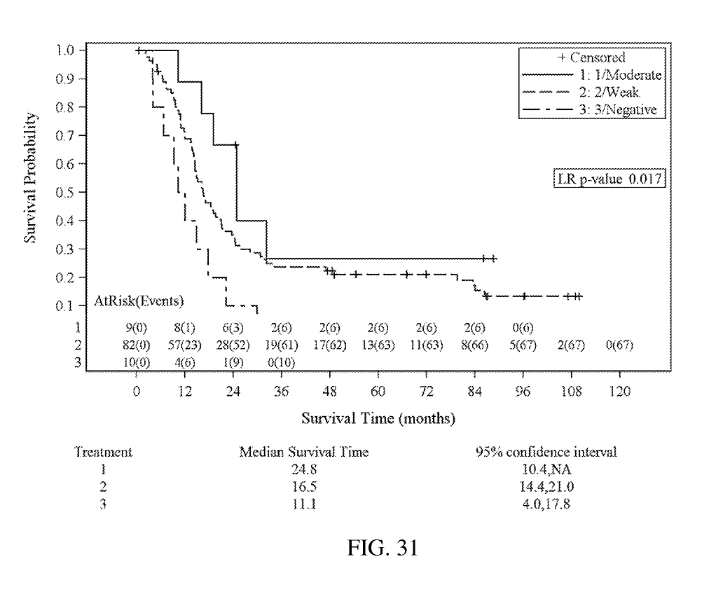

FIG. 31 illustrates that in gemcitabine-treated patients, the subgroup with negative hENT1 expression exhibited shorter OS as compared to the subgroups with some detectable hENT1 expression (Weak, Moderate or Strong).

### Example 10

### hENT1 Endpoint as Determined by No hENT1 with Combination Algorithm

A hENT1 endpoint was calculated and used to correlate hENT1 expression with the overall survival (OS) of patients from the RTOG 9704 study.

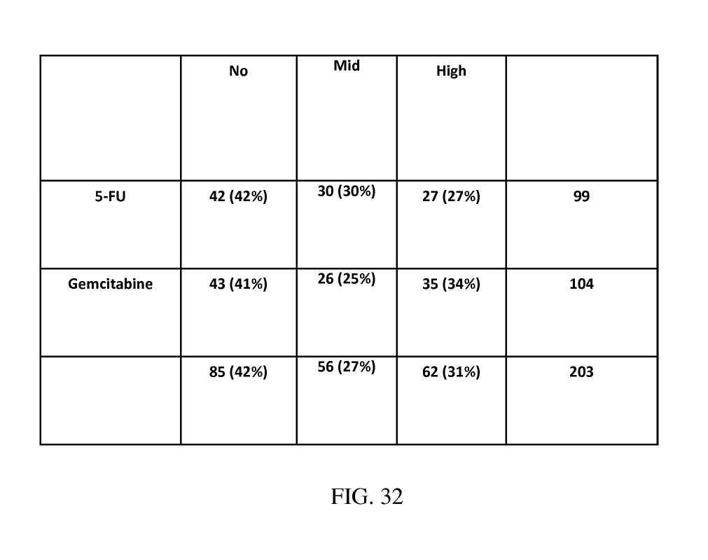

Preliminary results obtained from the RTOG 9704 study and analyzed for hENT1 endpoint by Method Six are set forth in FIGS. 32-34.

FIG. 32 depicts the frequency of patients across the 3 subgroups of hENT1 expression defined categorically as High, Mid, and No, where a sample is defined using method six, which combines magnification and percent staining. A sample is defined as High when the sample has the criterion based on the exemplary scoring sheet of FIG. 2 of Yes for ≧50% tumor cells membrane resolvable at 10×. A sample is defined as Mid when the sample has the criterion of Yes for membranes resolvable at 10× and No for ≧50% tumor cells membrane resolvable at 10×. And a sample is defined as No when the sample has the criterion of No for membranes resolvable at 10× and No for ≧50% tumor cells membrane resolvable at 10×. If multiple samples were obtained from a patient, if any of the samples were classified as High, then that patient is classified as High. Patients having No hENT1 as defined by this algorithm can be administered a gemcitabine derivative.

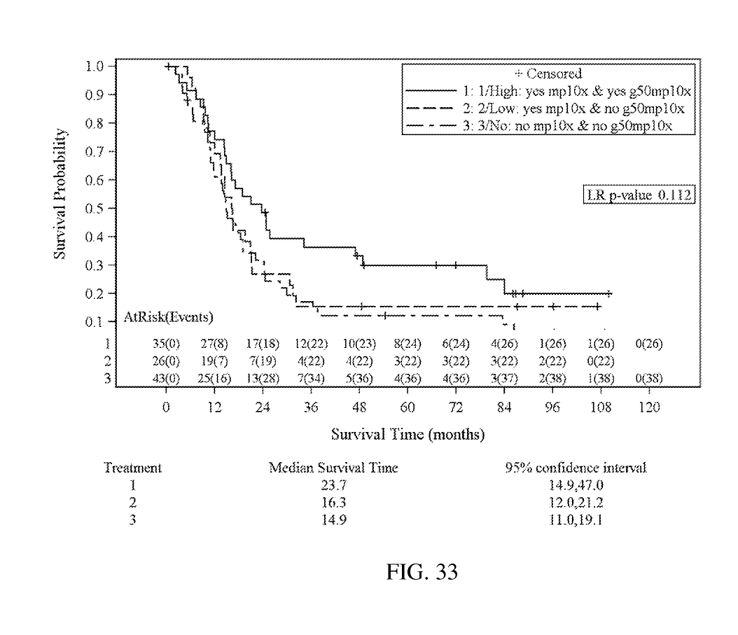

FIG. 33 illustrates that in gemcitabine-treated patients, the subgroup with No hENT1 expression exhibited the shortest OS followed by the Mid hENT1 subgroup and the High hENT1 subgroup had the longest OS.

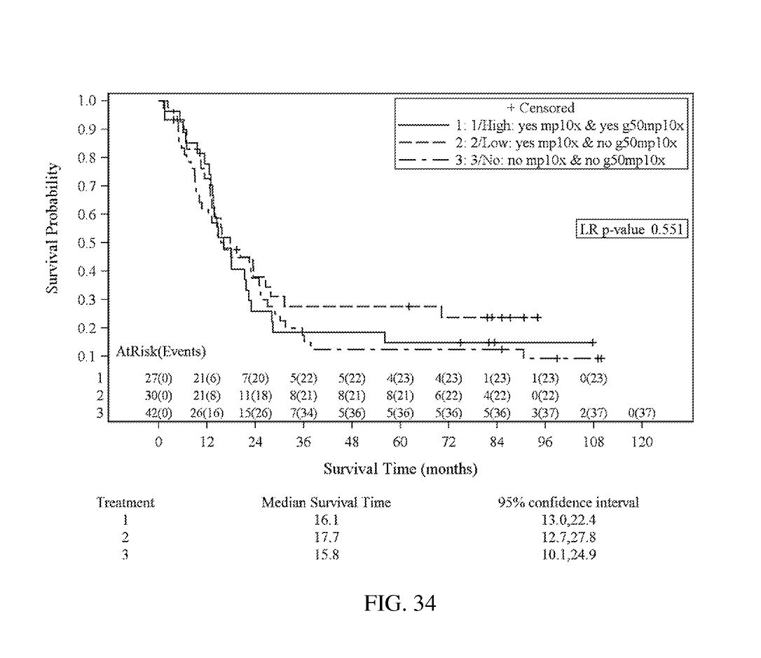

In contrast, FIG. 34 shows that 5FU treated patients do not demonstrate improvement in OS between the High, Mid and No hENT1 subgroups of patients.

### Example 11

The following table contains the cut-off for the H-score, percentage of positive tumor staining, magnification and a combination algorithm that incorporates both the percentage of positive tumor staining and the magnification.

TABLE 2
Association Between hENT1 Cut-off and Overall Survival

| | | HR (CI) | Median | Median | % of | HR for | HR for |
|---|---|---|---|---|---|---|---|
| | | for hENT1- | OS in | OS in | Patients | Gem vs 5- | Gem vs 5- |
| | | Low vs - | Gem | Gem | Categorized | FU in | FU in |
| | | High in | hENT1- | hENT1- | as | hENT1- | hENT1- |
| Staining | Definition of | Gem | Low | High | hENT1- | Low | High |
| Variable | Low Group | Patients | Patients | Patients | Low | Patients | Patients |
| H-score | H-score <50 | 0.63 | 14.2 | 19.6 | 33% | 1.35 | 0.86 |
| | | (0.40-0.98) | | | | (0.83, 2.20) | (0.58, 1.27) |
| 50% | >50% of tumor is | 0.61 | 14.2 | 19.6 | 35% | 1.37 | 0.84 |
| tumor | negative | (0.39-0.95) | | | | (0.85, 2.21) | (0.57, 1.25) |
| staining | | | | | | | |
| Magnification | Unequivocal | 0.67 | 14.8 | 21.0 | 55% | 1.14 | 0.87 |
| (intensity) | membranes | (0.43-1.04) | | | | (0.77, 1.69) | (0.54, 1.41) |
| | visible only at | | | | | | |
| | 20x, 40x or | | | | | | |
| | negative | | | | | | |
| Magnification | Unequivocal | 0.58 | 14.8 | 24.2 | 63% | 1.20 | 0.77 |
| and | membranes | (0.37-0.92) | | | | (0.82, 1.74) | (0.46, 1.30) |
| % tumor | visible only at | | | | | | |
| staining | 20x, 40x or | | | | | | |
| | negative OR | | | | | | |
| | >=50% of tumor | | | | | | |
| | is negative | | | | | | |

For each of the hENT1 cut-offs the median overall survival for gemcitabine-treated patients is longer for hENT1-high patients as compared to hENT1-low patients. In addition, the overall survival is longer for gemcitabine-treated patients as compared to 5-FU-treated patients in the hENT1-high subgroup (hazard ratio<1). Further, in the hENT1-low subgroup the overall survival for 5-FU-treated patients is longer as compared to the gemcitabine-treated patients (hazard ratio>1).

In addition to demonstrating a difference in overall survival for gemcitabine-treated patients, the cut-off between hENT1-high and -low, needs to be robust and reproducible. Therefore, a chosen cut-off scoring method is based on the combination of the magnification and percent of positive tumor.

### Example 12

### A Phase II Clinical Trial Comparing Gemcitabine-5′-Elaidate With Gemcitabine as First Line Therapy in Patients With Metastatic Pancreatic Adenocarcinoma

To compare effectiveness of gemcitabine-5′-elaidate and gemcitabine in patients having varying levels of hENT1 expression, a clinical trial is being conducted in which patients with metastatic pancreatic adenocarcinoma (stage 4) are tested to determine the hENT1 expression level of the cancer cells and are treated with either gemcitabine-5′-elaidate or gemcitabine as a first line therapy.

The primary objective of the trial will be to compare the efficacy of gemcitabine-5′-elaidate and gemcitabine in patients with metastatic pancreatic carcinoma and low hENT1 expression. Secondary objectives include the following: compare the efficacy of gemcitabine-5′-elaidate and gemcitabine in patients with known hENT1 status (all patients and high hENT1 expression); compare the tolerability and toxicity of gemcitabine-5′-elaidate with gemcitabine; compare changes in pain severity in patients receiving gemcitabine-5′-elaidate and gemcitabine; compare changes in health status in patients receiving gemcitabine-5′-elaidate and gemcitabine; perform sparse pharmacokinetic (PK) sampling in patients taking gemcitabine-5′-elaidate to contribute towards development of a population PK model of gemcitabine-5′-elaidate; and evaluate the clinical utility of the hENT1 diagnostic test.

The primary objective or endpoint is measured by overall survival (OS) in patients with low hENT1 expression. Secondary endpoints are measured by OS in all patients and in patients with high hENT1 expression; objective tumor response rate (ORR), duration of response, and progression-free survival (PFS) in patients with measurable/evaluable disease using RECIST 1.1; CA 19-9 velocity and response rate; drug tolerability and toxicity using clinical adverse events (AE) monitoring, clinical laboratory testing, ECG outcomes, and dose modifications of protocol-specified treatment; change from baseline in pain severity measured by the worst pain on the Brief Pain Inventory (BPI) short form; change from baseline in health status measured by the Euroquol EQ-5D instrument and EQ VAS form; and PK profile of gemcitabine-5′-elaidate based on sparse sampling.

Approximately 360 patients from about 90 investigation centers throughout Europe, Australia and the Americas will participate. Gemcitabine-5′-elaidate and gemcitabine will be randomized without regard to hENT1 expression status. Patients are deemed eligible if the following criteria are met: they have been diagnosed with metastatic pancreatic ductal adenocarcinoma (stage 4); there has been histological/cytological confirmation of metastatic tissue (not primary tumor) by a central pathology laboratory to ensure sufficient material is available for later hENT1 analysis; any adjuvant chemotherapy or radiotherapy (if administered) must have been administered more than at least 6 months prior to randomization; palliative radiotherapy (if administered) must have occurred more than at least 1 month prior to randomization; a CT scan must have been performed less than at least 30 days prior to randomization; the patient must have a ECOG performance status of 0 or 1; the patient's estimated life expectancy must be greater than or equal to 12 weeks; the patient must be greater than or equal to 18 years of age; the patient must have adequate hematological and biological function, including bone marrow function, hepatic function and renal function; the patient must provide written consent on an Institutional Review Board/Institutional Ethics Committee (IRB/IEC)-approved Informed Consent Form prior to any study-specific evaluation.

Exclusion criteria for patients include: prior palliative chemotherapy for pancreatic cancer; radical pancreatic resections (e.g., Whipple procedure) less than 6 months prior to randomization; exploratory laparotomy, palliative (e.g., bypass) surgery, or other procedures (e.g., stents) less than 14 days prior to randomization; symptomatic brain metastases; participation in other investigational drug clinical studies less than that at least 30 days prior to randomization; concomitant treatment with prohibited medications (e.g., concurrent anticancer therapy including other chemotherapy, radiation, hormonal treatment, or immunotherapy) less than at least 30 days prior to randomization; history of allergy to gemcitabine or eggs; presence of any serious or unstable concomitant systemic disorder incompatible with the clinical study (e.g., substance abuse, uncontrolled intercurrent illness including active infection, arterial thrombosis, symptomatic pulmonary embolism); any disorder that would hamper protocol compliance; prior nonpancreatic malignancy treated with chemotherapy; prior malignancies treated with surgery or radiotherapy alone that have not been in remission more than at least 3 years; females who are pregnant or breastfeeding; refusal to use adequate contraception for fertile patients (females and males during the study and for 6 months after the last protocol-specified treatment); and any other reason the investigator considers that the patient should not participate in the study.

Eligible patients will be randomized (1:1) using an Interactive Voice/Web Response System (IVRS/IWRS) to receive either gemcitabine-5′-elaidate or gemcitabine. Randomization will be prospectively stratified by Eastern Cooperative Oncology Group (ECOG) performance status (0 vs. 1) and region (North America vs. Western Europe vs. Eastern Europe vs. South America vs. Australia). Each cycle of gemcitabine-5′-elaidate will be administered weekly for 3 weeks every 4 weeks (4th week rest). The first cycle of gemcitabine comprises weekly administration for 7 weeks (8th week rest); subsequent cycles comprise weekly administration for 3 weeks every 4 weeks in accordance with manufacturer's labeling. Dosing will be delayed or decreased according to protocol-specified toxicity criteria. No dose escalation beyond the starting dose is allowed. Gemcitabine-5′-elaidate and gemcitabine will be infused intravenously over 30±3 min.

Protocol-specified treatment will continue until there is clinical tumor progression or unacceptable toxicity. Patients will undergo serial assessments for antitumor efficacy, drug safety, pain severity, and health status. Sparse blood sampling for population PK analyses will be conducted in all patients treated with gemcitabine-5′-elaidate. One specimen of blood will be collected and banked centrally for future pharmacogenetic evaluation of polymorphisms relating to drug metabolism and tumor outcomes. Central laboratories will confirm tumor type and adequacy of biopsy prior to randomization, although randomization will proceed in a hENT1-blind manner. Tumor hENT1 status will be determined after randomization but before the final efficacy analysis so that the primary endpoint (overall survival in hENT1-low patients) can be assessed prospectively, using predefined criteria to classify patients as hENT1-high or -low. Genome-wide RNA profiling will be performed on tumor specimens to assess expression signatures associated with clinical response to study drugs. Proteomics will also be performed on serum/plasma to identify relationships between efficacy and study drug. Central/core laboratories will be used for hematology and chemistry, as well as Cancer Antigen 19-9 (CA 19-9), ECG interpretation, pharmacogenomics, proteomics and PK assay. Investigational centers will interpret tumor scans locally for the purpose of making treatment decisions and for final tumor response evaluation. When possible, relapsing patients will undergo tumor biopsy before second-line therapy is initiated.

Adverse events (AEs) will be assessed from the time informed consent is obtained through 28 days after the last protocol-specified treatment administration. Patients with stable disease or better will continue to have tumor scans every 8±1 weeks until tumor progression. All patients will be followed indefinitely at approximately monthly intervals to determine survival status. After discontinuation of protocol-specified treatment, second-line and subsequent specific anticancer therapy used at the investigator's discretion will be documented on the electronic Case Report Form (eCRF). Patients randomized to gemcitabine may not cross over to receive gemcitabine-5′-elaidate.

The primary endpoint is overall survival (OS) in patients with low hENT1 expression. If the hENT1 expression data are available for analysis prior to randomizing 360 patients then an interim analysis for sample size re-estimation may be performed.

An independent data monitoring committee (IDMC) will monitor the conduct of the study. The IDMC will review safety data from the study with a frequency sufficient to adequately assess patient safety, and may at any time request any data it feels are warranted. During the study, access to the information provided to the IDMC will be limited to IDMC and sponsor designated personnel who support the IDMC.

FIG. 35 illustrates the Study Schema of the clinical trial. The schema summarizes planned periods for randomization, treatment, response evaluation and follow-up, and drug cycle dosing.

As indicated in FIG. 35, the forms of administration are gemcitabine-5′-elaidate for infusion 15 mg/mL, and commercially-available gemcitabine HCl for injection. Treatment is to be initiated within 3 days after randomization and continued until tumor progression or unacceptable toxicity. Gemcitabine-5′-elaidate and gemcitabine are to be administered as 30±3 min intravenous infusions via a peripheral vein (or central venous catheter) under medical supervision. The dose of gemcitabine-5′-elaidate is 1250 mg/m2/day on Day 1, 8, and 15 in 4 week schedules (i.e., Day 1, 8, 15, q4w). The dose of gemcitabine is 1000 mg/m2/day weekly for 7 weeks in an initial 8 week schedule (i.e., q8w) followed by Day 1, 8, and 15 in 4 week schedules (i.e., Day 1, 8, and 15, q4w) for subsequent cycles (in accordance with the manufacturer's labeling).

Dose modification criteria are to be administered on prescribed days±1 day. In the case of significant drug-related toxicity, the dose will be either delayed or omitted until the patient has sufficiently normalized. When a dose cannot be given within a window due to toxicity, then that dose will be omitted and the next scheduled dose will be administered on time. Criteria for reduction of dose will include absolute neutrophil count, platelet count and other indicia. If a treatment cycle has been delayed due to drug-related toxicity, and the delay is greater than 3 weeks, treatment will be permanently discontinued.

Withdrawal criteria include the following: disease progression (based on tumor scan or clinical status); intercurrent illness that prevents administration of protocol-specified treatment; unacceptable toxicity; patient withdrawal of consent to further treatment; major noncompliance that may affect patient safety; pregnancy; and investigator decision. Patients who withdraw will remain in the study and will be followed for safety (up to 28 days after last dose), for disease progression (every 8±1 weeks until disease progression), and for survival status (at approximately monthly intervals until death).

Statistical analyses will include the following populations: intent-to-treat population (all randomized patients); tumor-evaluable population (all patients who received at least one dose of protocol-specified treatment who have measurable tumor lesions and known hENT1 status); CA 19-9-evaluable population (all patients who received at least one dose of protocol-specified treatment who have a baseline CA 19-9 level>1.5×ULN, at least one postbaseline CA 19-9 level, and known hENT1 status); CTC-evaluable population (all patients who received at least one dose of protocol-specified treatment who have a baseline CTC value, at least one postbaseline CTC value, and known hENT1 status); and safety population (all patients who received at least one dose of protocol-specified treatment).

The prognostic utility of the hENT1 expression diagnostic test will be evaluated by comparing the primary and secondary endpoints in hENT1 low patients to that of hENT1 high patients. The comparison of hENT1 Low and High patients will be performed separately for the gemcitabine-treated patients, gemcitabine-5′-elaidate—treated patients, and all patients with known hENT1 status.

While preferred aspects of the present invention have been shown and described herein, it will be obvious to those skilled in the art that such aspects are provided by way of example only. Numerous variations, changes, and substitutions will now occur to those skilled in the art without departing from the invention. It should be understood that various alternatives to the aspects of the invention described herein may be employed in practicing the invention. It is intended that the following claims define the scope of the invention and that methods and structures within the scope of these claims and their equivalents be covered thereby.

1. A method for determining whether a gemcitabine derivative that is capable of being transported into the tumor tissue by a mechanism that is independent of the hENT1 protein is suitable for administration to a patient with cancer, comprising conducting an immunohistochemistry assay on a biological sample comprising cancer cells or cancer cell proteins from said patient to determine whether the hENT1 protein level is classified as Low, wherein said Low classification meets the criterion of having less than 50% of the cancer cells display hENT1 membrane staining with a hENT1 antibody upon examination with a 10× ocular of a light microscope, wherein if said immunohistochemistry assay indicates that the level of hENT1 protein is Low, the gemcitabine derivative that is capable of being transported into the tumor tissue by a mechanism that is independent of the hENT1 protein is suitable for administration to a patient with cancer.

2. The method of claim 1, wherein the cancer is pancreatic adenocarcinoma.

3. The method of claim 1, wherein said gemcitabine derivative is transported through the plasma membrane by a mechanism that does not utilize the hENT1 transporter.

4. The method of claim 3, wherein said mechanism of transport is by passive diffusion across the membrane.

5. A method of classifying hENT1 biomarker expression in a tissue sample comprising:
a) obtaining a tissue sample from a cancer patient;
b) visualizing hENT1 biomarker protein expression in the tissue sample using immunohistochemical staining with an anti-hENT1 antibody;
c) determining hENT1 protein staining intensity in the tissue sample; and
d) classifying hENT1 biomarker expression as LOW when less than 50% of the tissue sample displays hENT1 membrane staining with an anti-hENT1 antibody upon examination with a 10× ocular of a light microscope.

6. The method according to claim 5, wherein the tissue sample is from a pancreatic adenocarcinoma.

7. A method of identifying a pancreatic cancer patient suitable for treatment with a gemcitabine derivative, comprising:
a) visualizing hENT1 protein expression in cancer cells in a biological sample from the patient by immunohistochemical staining using an anti-hENT1 antibody; and
b) assigning a protein expression classification of LOW when less than 50% of the cancer cells display hENT1 membrane staining upon examination with a 10× ocular of a light microscope; and
c) identifying patients exhibiting LOW hENT1 protein expression as being suitable for treatment with a gemcitabine derivative.

8. The method of claim 7, wherein the cancer cells are in the form of a tissue sample isolated from the patient.

9. The method of claim 7, wherein the tissue sample is from a metastatic pancreatic tumor.

10. The method of claim 7, wherein the patient was not exposed to gemcitabine prior to isolation of the tissue sample.

11. A method of predicting a pancreatic cancer patient's responsiveness to gemcitabine therapy comprising:
a. visualizing hENT1 biomarker protein expression in a tissue sample from the pancreatic cancer patient using immunohistochemical staining with an anti-hENT1 antibody;
b. determining hENT1 protein staining intensity in the tissue sample;
c. classifying hENT1 biomarker expression as LOW when less than 50% of the tissue sample displays hENT1 membrane staining with an anti-hENT1 antibody upon examination with a 10× ocular of a light microscope; and
d. predicting a poor response to gemcitabine therapy when the patient's tissue sample is classified as having LOW hENT1 biomarker expression.

12. The method of claim 11, wherein the tissue sample is obtained from a metastatic pancreatic tumor.

13. A method of stratifying cancer patients for overall survival comprising:
a. staining a biological sample comprising tumor tissue of each cancer patient with an anti-hENT1 antibody;
b. visually detecting antibody bound to the tumor tissue;
c. scoring antibody staining intensity; and
d. classifying the cancer patient into a LOW hENT1 expression subgroup when greater than 50% of the tumor tissue is scored as negative for hENT1 membrane staining with a membrane intensity of 0; and
e. stratifying the LOW hENT1 expression subgroup as having shorter overall survival as compared with subgroups that are not classified as exhibiting LOW hENT1 expression.
